(19) United States
(12) Patent Application Publication
(10) Pub. No.: US 2002/0099035 A1
(43) Pub. Date: Jul. 25, 2002

(54) Method for preparing alpha-sulfonyl hydroxamic acid derivatives

(76) Inventors: Vincent P. Sandanayaka, Wesley Hills, NY (US); Arie Zask, Ney York, NY (US); Aranapakam M. Venkatesan, Rego Park, NY (US); Jannie L. Baker, Hardeeville, SC (US); Lalitha Krishnan, Suffern, NY (US); Sreenivasulu Megati, New City, NY (US); Joseph Zeldis, New City, NY (US)

Correspondence Address: Egon E. Berg, American Home Products Corporation, Patent Law Department - 2B, One Campus Drive, Parsippany, NJ, 07054, US

(21) Appl. No.: 09/769,107

(22) Filed: Jan. 24, 2001

(51) Int. Cl. C07F 9/06; C07F 9/28; A61K 31/675; A01N 57/00; C07D 211/36; C07D 211/40; C07D 211/54; A61K 31/695; A61K 31/66

(52) U.S. Cl. 514/89; 546/242; 546/22; 546/216; 514/63; 514/127; 514/227.5; 514/238.8; 514/252.12; 514/315; 514/408; 514/557; 514/575; 514/625; 514/451

(57) ABSTRACT

Compounds of the formula:

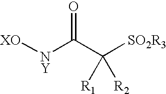

that can be important as matrix metalloproteinase (MMP) and TNF-alpha converting enzyme (TACE) inhibitors, phosphodiesterase inhibitors, renin inhibitors, antithrombotics, and 5-lipoxygenase inhibitors are prepared by novel methods of the present invention.

This application claims the benefit of U.S. application Ser. No. 09/492,975 and U.S. Provisional Application No.(Not Yet Known), filed Jan. 27, 2000 and is a continuation-in-part of that prior application which is incorporated by reference herein in its entirety.

### FIELD OF THE INVENTION

The invention relates to a novel method of producing alpha-sulfonyl hydroxamic acid derivatives that can be important as matrix metalloproteinase (MMP) and TNF-alpha converting enzyme (TACE) inhibitors, phosphodiesterase inhibitors, renin inhibitors, antithrombotics, and 5-lipoxygenase inhibitors. This invention also relates to new alpha-sulfonyl hydroxamic acid derivatives and their preparation by the new process and pharmaceutical compositions containing them

### BACKGROUND OF THE INVENTION

Matrix metalloproteinases are a family of structurally related zinc-containing enzymes that mediate the breakdown of the extracellular matrix proteins. Members of this family, which include collagenases, stromelysins, and gelatinases are involved in the normal tissue remodeling process such as wound-healing, angiogenesis, and pregnancy. In these pathological processes, the MMP activity is tightly regulated by the endogenous tissue inhibitors of matrix metalloproteinases (TIMPS). In pathological conditions, this fine balance between MMP-TIMP can be disrupted leading to several disease states including rheumatoid and osteoarthritis, atherosclerosis, tumor growth, metastasis, and fibrosis. Therapeutic inhibition of MMPs is a promising approach for treatment of these diseases and therefore the MMPs are attractive targets for rational drug design.

TACE is also a new member of metalloproteinase family, which catalyses the formation of tumor necrosis factor-alpha precursor protein. TNF-alpha was selected as one of the early targets leading to the successful cloning and sequencing of human TNF-alpha in 1984 by Goeddel and collegues. TNF-alpha is a very powerful proinflammatory mediator produced by activated macrophages, blood monocytes, and mast cells. In addition to its anti-tumor properties, TNF-alpha is a proinflammatory cytokine that has a central role in rheumatoid arthritis, and Crohn's disease. Animal models and association studies in humans have indicated a potential role for TNF in insulin resistance, multiple sclerosis, organ failure, pulmonary fibrosis, and HIV infection. Therefore, the inhibition of TNF-alpha has been the focus of drug discovery.

The lipoxygenases are a family of enzymes, which catalyze the oxygenation of arachidonic acid leading to the production of leukotrienes. Leukotrienes have been implicated as important mediators in asthma, rheumatoid arthritis, gout, psoriasis, allergic rhinitis, adult respiratory distress syndrome, Crohn's disease, endotoxin shock, and inflammatory bowel disease. It is believed that inhibition of these enzymes will provide effective systematic treatment of these diseases. Renin inhibitors can be used to control or prevent high blood pressure and cardiac insufficiency.

Alpha-sulfonyl hydroxamic acids of the general formula I have been disclosed as potent MMP and TACE inhibitors (Venkatesan, A. M.; Grosu, G. T.; Davis, J. M.; Baker, J. L.; Levin, J. I. PCT Int. Appl. WO 9942436; Barta, T. E.; Becker, D. P.; Boehm, T. L.; De Crescenzo, G. A.; Villamil, C. I.; McDonald, J. J.; Freskos, J. N.; Getman, D. P. PCT Int. Appl. 9925687; Almstead, N. G.; Bookland, R. G.; Taiwo, Y. O.; Bradley, R. S.; Bush, R. D.; De B.; Natchus, M. G.; Pikul, S. PCT Int. Appl. 9906340; Venkatesan, A. M.; Grosu, G. T.; Davis, J. M.; Baker, J. L.; Hu, B.; O'Dell, M. J.; Cole, D.C.; Jacobson, M. P., PCT Int. Appl. WO 9838163; Venkatesan, A. M.; Grosu, G. T.; Davis, J. M.; Baker, J. L. PCT Int. Appl. WO 9837877; Levin, J. I.; Venkatesan, A. M.; Zask, A.; Sandanayaka, V. P.; U.S. Ser. No. 09/492686 filed; Jan. 27, 2000; Zook, S. E.; Dagnino, R.; Deason, M. E.; Bender, S. L.; Melnick, M. PCT Int. Appl. WO 9720824), renin inhibitors (Branca, Q.; Heitz, M. P.; Neidhart, W.; Stadler, H.; Vieira, E.; Wostl, W. EP 509354), 5-lipoxygenase inhibitors (Brooks, D. W.; Summers, J. B.; Rodriques, K. E.; Maki, R. G.; Dellaria, J. F.; Holms, J. H.; Moore, J. L. U.S. Pat. No. 5,250,565), and antithrombotics (Nakane, M.; Reid, J. U.S. Pat. No. 4,734,425).

General preparation of alpha-sulfonylhydroxamates in the above literature involves first, the alkylation of appropriately substituted mercaptan derivative with either substituted or unsubstituted alpha-bromoacetic acid ester to give alpha-thio ester followed by oxidation of sulfur to sulfone to provide alpha-sulfonyl ester. This alpha-sulfonyl ester is converted to the corresponding hydroxamic acid derivative via the carboxylic acid. Alternatively, the enolate of the carbonyl compound is treated with the appropriately substituted disulfide to obtain the alpha-thio ester, which is then oxidized to the corresponding sulfone. The alpha-sulfonyl ester is converted to the hydroxamic acid derivative as mentioned above. In either case, the preparation of the thiol or the disulfide requires multiple steps that involve sulfonyl chloride, protected thiols, or disulfides as intermediates and the oxidation step to convert alpha-thio ester to alpha-sulfonyl ester.

It is the object of this invention to provide a novel method for preparing alpha-sulfonyl hydroxamic acid derivatives, which provides the target molecules in a highly convergent and efficient manner.

### SUMMARY OF THE INVENTION

In accordance with the present invention is provided a method of preparing alpha-sulfonyl hydroxamic acid derivatives of the formula I:

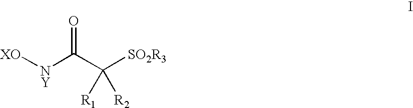

wherein

X is hydrogen, alkyl of 1-6 carbon atoms, benzyl, hydroxyethyl, t-butyldimethylsilyl, trimethylsilyl or tetrahydropyranyl;

Y is hydrogen, alkyl of 1-6 carbon atoms, aryl of 6 to 10 carbon atoms, 5-10 membered heteroaryl having 1-3 heteroatoms selected from N, NR₄, O and S, cycloalkyl of 3-6 carbon atoms, 5-10 membered cycloheteroalkyl; wherein said alkyl, aryl, heteroaryl, cycloalkyl and cycloheteroalkyl group of Y is optionally substituted on any atom capable of substitution, with 1 to 3 substituents selected from the group consisting of halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅ perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;

R₁ and R₂ are each, independently, hydrogen; aryl of 6 to 10 carbon atoms; 5-10 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms having 1 to 3 double bonds; alkynyl of 2-18 carbon atoms having from 1 to 3 triple bonds; or R₁ and R₂ taken together with the carbon atom to which they are attached form a cycloalkyl ring of 3-8 carbon atoms or a 5-10 membered cycloheteroalkyl ring; and wherein the aryl, heteroaryl, cycloalkyl, cycloheteroalkyl, alkyl, alkenyl, and alkynyl, may be optionally substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅ perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;

R₃ is alkyl of 1-18 carbon atoms, alkenyl of 2-18 carbon atoms having 1 to 3 double bonds, alkynyl of 2-18 carbon atoms having from 1 to3 triple bonds, cycloalkyl of 3-6 carbon atoms, 5-10 membered cycloheteroalkyl, aryl of 6 to 10 carbon atoms, 5-6 membered heteroaryl having 1-3 heteroatoms selected from N, NR₄, O, and S; wherein said alkyl, alkenyl, alkynyl, cycloalkyl, cycloheteroalkyl, aryl and heteroaryl of R₃ may optionally be substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅ perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;

R₄ is hydrogen; aryl; aralkyl, heteroaryl; heteroaralkyl, alkyl of 1-6 carbon atoms; cycloalkyl of 3-6 carbon atoms; —C(O)ₙR₅, —CONR₅R₆ or SO₂R₅;

R₅ and R₆ are each independently hydrogen, optionally substituted aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms or alkynyl of 2-18 carbon atoms; or R₅ and R₆ taken together with the nitrogen atom to which they are attached may form a 5-10 membered cycloheteroalkyl ring; and

n is 1 or 2; or pharmaceutical salts thereof, comprising the steps of reacting a sulfonyl fluoride of the formula III

R₃′SO₂F III

wherein R₃′ is as hereinabove defined for R₃ with the proviso that R₃′ does not contain a group that can form an anion under basic conditions;

with a carbonyl compound of the formula IV:

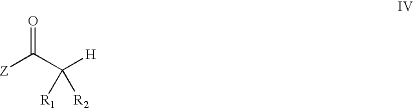

wherein Z is H, OH, YNOX, or OR₅, and X, Y, R₁, R₂, R, and R₆ are as hereinabove defined; in the presence of a metal hydride or amide base in an ether organic solvent at temperatures from about −78° C. to about room temperature (eg up to about 15° C. to about 30° C., preferably up to about 20-25° C.) to produce an alpha-sulfonyl carbonyl compound of formula V:

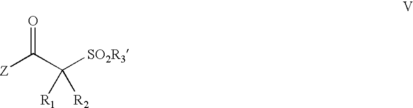

wherein Z, R₁, R₂ and R₃′ are as hereinabove defined; and converting compound of formula V into a hydroxamic acid derivative.

Compounds of Formula I may also be prepared by reacting a sulfonyl fluoride of formula III:

R₃′SO₂F III

wherein R₃′ is as hereinabove defined for R₃ with the proviso that R₃′ does not contain a group that can form an anion under basic conditions; with an enol ether of formula VIII:

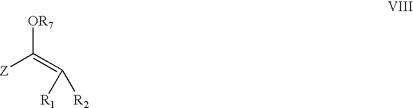

wherein

Z is H, OH, YNOX, or OR₅, and R₁ and R₂, are as hereinabove defined;

R₇ is cycloalkyl of 3-6 carbon atoms; 5-10 membered cycloheteroalkyl; alkyl of 1-18 carbon atoms; alkenyl of 2-18 carbon atoms having from 1 to 3 double bonds; alkynyl of 2-18 carbon atoms having from 1 to 3 triple bonds; or —SiR₈R₉R₁₀; and

R₈, R₉, and R₁₀ are each, independently, aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms having from 1 to 3 double bonds; alkynyl of 2-18 carbon atoms having from 1 to 3 triple bonds; or two of R₈, R₉, and R₁₀ taken together with the silicon atom to which they are attached form a heterocyclic ring of 5 or 6 members;

in the presence of a Lewis acid or fluoride reagent in an ether organic solvent at temperatures ranging from about −78° C. to about room temperature (eg up to about 15-30° C. to produce an alpha-sulfonyl carbonyl compound of formula V:

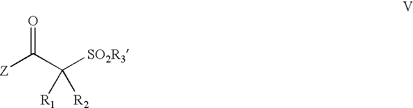

wherein Z, R₁, R₂ and R₃′ are as hereinabove defined, and converting the compound of Formula V into a hydroxamic acid derivative.

In other embodiments of the present invention are provided methods of preparing alpha-sulfonyl hydroxamic acid derivatives of the general formula I:

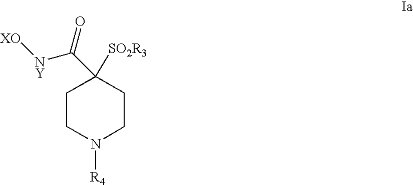

wherein

X is hydrogen, or alkyl of 1-6 carbon atoms;

Y is hydrogen, alkyl of 1-6 carbon atoms, aryl of 6 to 10 carbon atoms, 5-10 membered heteroaryl having 1-3 heteroatoms selected from N, NR₄, O and S, cycloalkyl of 3-6 carbon atoms, 5-10 membered cycloheteroalkyl; wherein said alkyl, aryl, heteroaryl, cycloalkyl and cycloheteroalkyl group of Y is optionally substituted on any atom capable of substitution, with 1 to 3 substituents selected from the group consisting of halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅ perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR5R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;

R₃ is alkyl of 1-18 carbon atoms, alkenyl of 2-18 carbon atoms having 1 to 3 double bonds, alkynyl of 2-18 carbon atoms having from 1 to 3 triple bonds, cycloalkyl of 3-6 carbon atoms, 5-10 membered cycloheteroalkyl, aryl of 6 to 10 carbon atoms, 5-6 membered heteroaryl having 1-3 heteroatoms selected from N, NR₄, O and S; wherein said alkyl, alkenyl, alkynyl, cycloalkyl, cycloheteroalkyl, aryl and heteroaryl of R₃ may optionally be substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅ perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂) ₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;

R₄ is hydrogen; aryl; aralkyl, heteroaryl; heteroaralkyl, alkyl of 1-6 carbon atoms; cycloalkyl of 3-6 carbon atoms; —C(O)ₙR₅, —CONR₅R₆ or SO₂R₅;

R₅ and R₆ are each independently hydrogen, optionally substituted aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms or alkynyl of 2-18 carbon atoms; or R₅ and R₆ taken together with the nitrogen atom to which they are attached may form a 5-10 membered cycloheteroalkyl ring; and

n is 1 or 2; or pharmaceutical salts thereof, comprising the steps of

a) treating a compound of formula

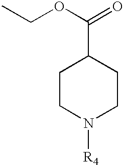

with diisopropylamide or lithium hexamethyldisilazide to form an enolate;

b) reacting the enolate with a sulfonyl fluoride of formula III:

R₃SO₂F III

to form a compound

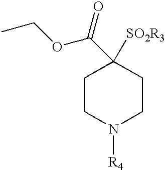

c) hydrolyzing the compound of step b) to produce

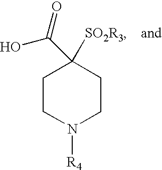

d) reacting compound of step c) with hydroxylamine or hydroxylamine derivative of the formula VII:

XONHY VII

in the presence of coupling reagent and polar organic solvent at temperatures ranging from 0° C. to about room temperature, eg up to about 15-30° C.

In other aspects of the invention are provided methods of preparing compounds of Formula 8

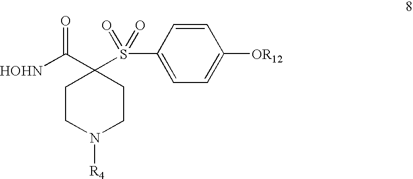

wherein

R₄ is hydrogen; aryl; aralkyl, heteroaryl; heteroaralkyl, alkyl of 1-6 carbon atoms; cycloalkyl of 3-6 carbon atoms; —C(O)ₙR₅, —CONR₅R₆ or SO₂R₅;

R₅ and R₆ are each independently hydrogen, optionally substituted aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms or alkynyl of 2-18 carbon atoms; or R₅ and R₆ taken together with the nitrogen atom to which they are attached may form a 5-10 membered cycloheteroalkyl ring; and

R₁₂ is methyl, n-butyl, 2-butynyl, or p-chlorophenyl;

and n is 1 or 2; or pharmaceutical salts thereof, comprising the steps of:

a) treating a compound of formula

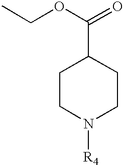

with diisopropylamide or lithium hexamethyldisilazide to form an enolate;

b) reacting the enolate with a sulfonyl fluoride of Formula 2:

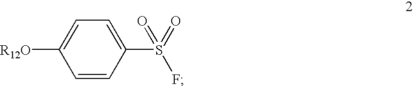

to form a compound of Formula 13

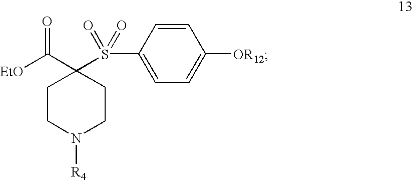

c) hydrolyzing compound of Formula 13 with lithium hydroxide to produce compound of Formula 14

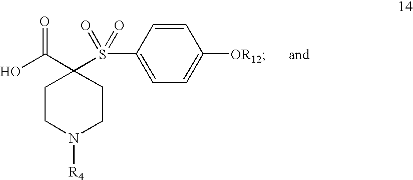

d) treating the compound of Formula 14 with oxalyl chloride, triethylamine, and hydroxylamine hydrochloride at temperatures ranging from 0° C. to about room temperature, eg up to about 15-30° C.

In some aspects of the present invention compounds of formula V are prepared

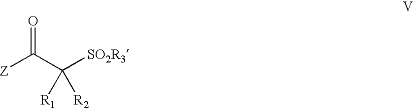

wherein

R₁, R₂ and R₃′ are as previously defined and Z is H, OH, YNOX, OR₅ or NR₅R₆, comprising reacting a sulfonyl fluoride of the formula III

R₃′SO₂F III

wherein R₃′ is as hereinabove defined;

with a carbonyl compound of the formula IV:

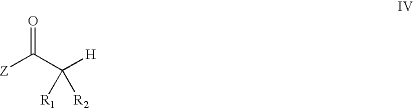

wherein Z, R₁ and R₂ are as previously defined, in the presence of a metal hydride or amide base in an ether organic solvent at temperatures from about −78° C. to about room temperature (eg up to from about 15° C. to about 30° C.).

Alternatively, compounds of Formula V are prepared

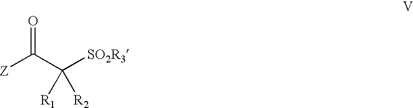

wherein

R₁, R₂ and R₃′ are as previously defined and Z is H, OH, YNOX, OR₅ or NR₅R₆, comprising reacting a sulfonyl fluoride of the formula III

R₃′SO₂F III

wherein R₃′ is as hereinabove defined with an enol ether of Formula VIII

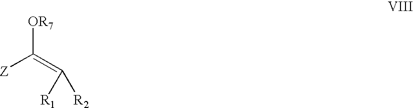

wherein

Z is H, OH, YNOX, OR₅ or NR₅R₆, and R₁ and R₂, are as hereinabove defined;

R₇ is cycloalkyl of 3-6 carbon atoms; 5-10 membered cycloheteroalkyl; alkyl of 1-18 carbon atoms; alkenyl of 2-18 carbon atoms having from 1 to 3 double bonds; alkynyl of 2-18 carbon atoms having from 1 to 3 triple bonds; or —SiR₈R₉R₁₀; and

R₈, R₉, and R₁₀ are each, independently, aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms having from 1 to 3 double bonds; alkynyl of 2-18 carbon atoms having from 1 to 3 triple bonds; or two of R₈, R₉, and R₁₀ taken together with the silicon atom to which they are attached form a heterocyclic ring of 5 or 6 members; in the presence of a Lewis acid or fluoride reagent in an ether organic solvent at temperatures ranging from about −78° C. to about room temperature (eg up to from about 15° C. to about 30° C.) to produce an alpha-sulfonyl carbonyl compound of formula V.

When Z is OR₅, compounds of Formula V may be converted to a hydroxamic acid derivative of Formula I in accordance with the steps of

reacting the alpha-sulfonyl carbonyl compound of the formula V with an alkali metal hydroxide in the presence of water, and/or ether organic solvent or alcohol at temperatures ranging from about 0° C. to about 100° C. to produce a carboxylic acid of the formula VI:

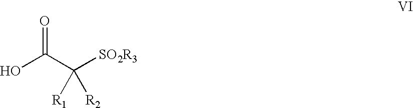

wherein, R₁, R₂, and R₃ are as hereinabove defined; and

reacting a carboxylic acid of formula VI with a hydroxylamine or hydroxylamine derivative of the formula VII:

XONHY VII

wherein X and Y are as hereinabove defined;

in the presence of suitable coupling reagent and polar organic solvent to produce a hydroxamic acid derivative of the formula I:

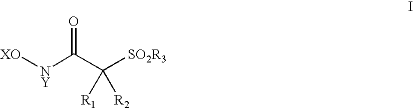

wherein X, Y, R₁, R₂, and R₃ are as hereinabove defined.

In other embodiments of the present invention, when Z is OH, compounds of Formula V may be converted to a hydroxamic acid derivative by reacting the alpha-sulfonyl carbonyl compound of formula V: with a hydroxylamine or hydroxylamine derivative of the formula VII:

XONHY VII

wherein X and Y are as hereinabove defined; in the presence of a coupling reagent and polar organic solvent at temperatures ranging from about 0° C. to about room temperature (eg up to from about 15° C. to about 30° C.).

Further, in accordance with the present invention sulfonyl fluoride compounds of Formula III can be prepared by reacting a sulfonyl chloride of formula II

R₃′SO₂Cl II

wherein R₃′ is as hereinabove defined for R₃ the proviso that R₃′ does not contain a group that can form an anion under basic conditions, with a fluorinating agent in the presence of a polar organic solvent at room temperature (eg at about 15° C. to about 30° C.) to produce a sulfonyl fluoride of formula III.

Further chemical transformations can be carried out before or after each step for compounds of the formula I, V, and VI in cases where R₁, R₂ or R₃ of the product differs from R₁, R₂ or R₃′ of the starting compound.

Groups which may form an anion under basic conditions of the invention and thus are excluded from the definition of R₃′, include, but are not limited to —OH, —NH, —SH, —COCH, —SO₂CH, —CHNO₂, CHCN. Accordingly, during the sulfonylation of the carbonyl compound, such substituents at R₃ should be avoided or protected and released later by deprotection, as designated by R₃′.

Sulfonyl chloride compounds of the present invention are commercially available or can be prepared by those skilled in the art in accordance with procedures described in the literature such as Kende, A. S.; Medoza, J. S., J. Org. Chem., 1990, 55, 1125-1126.

Appropriate fluorinating agents are taught, for example by Clark, J. H.; Hyde, A. J.; Smith, D. K. J.Chem.Soc.Chem.Comm. 1986, 791-792; Ichihara, J.; Matsuo, T.; Hanafusa, T.; Ando, T. J.Chem.Soc.Chem.Comm. 1986, 793-794 and include but are not limited to potassium fluoride, potassium fluoride-calcium fluoride mixture, or cesium fluoride.

Preferred ether organic solvents of the present invention are those known to those skilled in the art including, but not limited to tetrahydrofuran, diethylether or dioxane.

Bases used in methods of the present invention are those known to those skilled in the art, preferably metal hydride or amide bases, such as, but not limited to of lithiumdiisopropylamide, lithiumhexamethyldisilazide, and sodium hydride.

Lewis acids and fluoride reagents used in methods of the present invention are known to those skilled in the art and include, but are not limited to borontribromide, tetrabutylammonium and sodium hydride.

Polar organic solvents useful in methods of the present invention are known to those skilled in the art and include, but are not limited to acetonitrile, tetrahydrofuran and dimethylformamide.

Alkali metal hydroxides used in preferred methods of the present invention are known to those skilled in the art and include, but are not limited to lithium hydroxide and sodium hydroxide.

Alcohols used in some methods of the present invention are known to those skilled in the art and include, but are not limited to methanol and ethanol.

Coupling reagents of the present invention are those known to those skilled in the art including, but not limited one or more of 1-(3-dimethylaminopropyl)-3-ethylcarbodimide hydrochloride, N-hydroxybenzotriazole, N-methylmorpholine and oxalylchloride and triethylamine.

The present invention further relates to low molecular weight, non-peptide inhbitors of matrix metalloproteinases (MMPs) and TNF-alpha converting enzyme (TACE) for the treatment of rheumatoid arthritis, tumor metastasis, tissue ulceration, abnormal wound healing, periodontal disease, bone disease, diabetes and HIV infection.

Thus, in accordance with the present invention is provided compounds of Formula IX

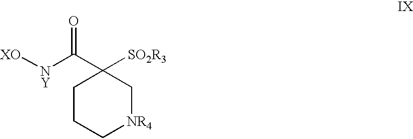

wherein:

X is hydrogen and alkyl of 1-6 carbon atoms; and

Y, R₃and R₄ are as previously defined, and pharmaceutical salts thereof.

Particularly preferred is 1-Benzyl-3-(4-methoxybenzenesulfonyl) piperidine-3-carboxylic acid hydroxamide, or pharmaceutical salts thereof.

Certain compounds prepared by the novel method of the present invention contain one or more asymmetric carbon atoms, giving rise to enantiomeric and diastereomeric forms of the compounds. In addition, certain compounds of this invention contain a carbon-carbon double bond, giving rise to cis- and trans-geometric isomers. It is to be understood that the invention encompasses the enantiomers, diastereomers, and geometrical isomers as well as mixtures thereof including racemic mixtures.

Alkyl, as used herein, refers to branched and straight chain alkyl groups, preferably having from 1 to 18 carbon atoms, and more preferably from 1 to 6 carbon atoms. Exemplary alkyl groups include methyl, ethyl, propyl, i-propyl, butyl, t-butyl, pentyl, hexyl, n-heptyl, octyl and the like.

Alkenyl, as used herein, refers to alkenyl groups, preferably having from 2-18 carbon atoms and more preferably from 2 to 6 carbon atoms, and having from 1 to 3 sites of alkenyl unsaturation (double bond).

Alkynyl, as used herein, refers to alkynyl groups, preferably having from 2-18 carbon atoms and more preferably from 2 to 6 carbon atoms, and having from 1 to 3 sites of alkynyl unsaturation (triple bond).

Cycloalkyl refers to cyclic alkyl groups of from 3 to 8 carbon atoms, and more preferably from 3-6 carbon atoms, including, by way of example, cyclopropyl, cyclobutyl, cyclopentyl, cyclohexyl, cyclooctyl and the like.

Heteroaryl, as used throughout, is a 5-10 membered mono- or bicyclic aromatic carbocyclic ring having from 1-3 heteroatoms selected from N, NR₄, S and O within the ring. Such heteroaryl groups can have a single ring (e.g. pyridyl or furyl) or multiple condensed rings (e.g. benzothienyl), which condensed ring may or may not contain a heteroatom. Heteroaryl is preferably

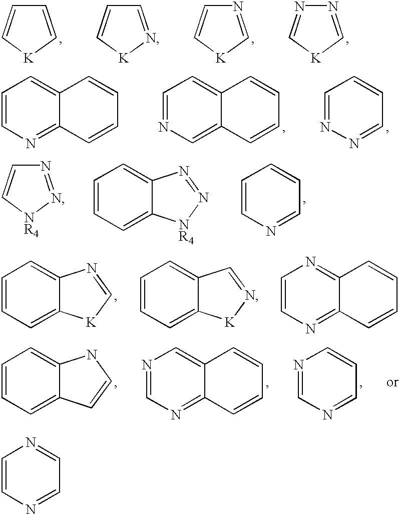

wherein K is defined as O, S or —NR₄, and R₄ is as hereinabove defined. More preferred heteroaryl rings include pyrrole, furan, thiophene, pyridine, pyrimidine, pyridazine, pyrazine, triazole, pyrazole, imidazole, isothiazole, thiazole, isoxazole, oxazole, indole, isoindole, benzofuran, benzothiophene, quinoline, isoquinoline, quinoxaline, quinazoline, benzotriazole, indazole, benzimidazole, benzothiazole, benzisoxazole, and benzoxazole. Heteroaryl groups of the present invention may have from 1 to 3 substituents, and more preferably may have one or two substituents.

5-10 membered cycloheteroalkyl is a saturated or unsaturated group having a single ring or multiple condensed rings, from 2 to 10 carbon atoms and from 1 to 3 heteroatoms selected from S, N, O, or NR₄ within the ring, wherein, in fused ring systems, one or more of the rings can be aryl or heteroaryl. Preferred cycloheteroalkyl are

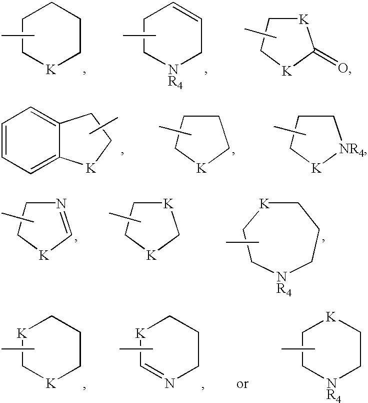

wherein K is O, N, S or NR₄; and R₄ is as hereinabove defined. The rings above shown as mono-radicals may also be illustrated as di-radicals eg when R₁ and R₂ together form a cycloheteroalkyl ring.

Preferred cycloheteroalkyl rings include piperidine, piperazine, morpholine, tetrahydropyran, tetrahydrofuran or pyrrolidine. Cycloheteroalkyl groups of the present invention may optionally be mono-, di- or tri substituted.

Aryl, as used herein refers to an unsaturated, aromatic carbocyclic group of from 6 to 10 carbon atoms having a single ring (phenyl) or multiple condensed rings (naphthyl), which condensed rings may or may not be aromatic. Preferred aryls include phenyl and naphthyl. Aryl groups may optionally be mono-, di- or tri-substituted.

Alkyl, alkenyl, alkynyl, and perfluoroalkyl include both straight chain as well as branched moieties. Alkyl, alkenyl, alkynyl, and cycloalkyl groups may be unsubstituted (carbons bonded to hydrogen or other carbons in the chain or ring) or may be mono- or poly-substituted.

Halogen means bromine, chlorine, fluorine, and iodine.

Suitable substituents of aryl, aralkyl, heteroaryl, heteroaralkyl, alkyl, alkenyl, alkynyl and cycloalkyl include, but are not limited to halogen, alkyl of 1-6 carbon atoms; alkenyl of 2-6 carbon atoms; alkynyl of 2-6 carbon atoms, cycloalkyl of 3-6 carbon atoms, —OR₅, —CN, —COR₅ perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl or 5-10 membered cycloheteroalkyl; and R₅ and R₆ are as hereinabove defined; —NR₅R₆ may form a cycloheteroalkyl ring such as pyrrolidine, piperidine, morpholine, thiomorpholine, oxazolidine, thiazolidine, pyrazolidine, piperazine or azetidine ring.

Pharmaceutically acceptable salts are those derived from pharmaceutically acceptable organic and inorganic acids such as lactic, citric, acetic, tartaric, succinic, maleic, malonic, hydrochloric, hydrobromic, phosphoric, nitric, sulfuric, methanesulfonic, and similarly known acceptable acids.

In the compounds referred to above, examples of Z are preferably OH or OR₅ for example where R₅ is alkyl (preferably 1-6 carbon atoms, e.g. methyl, ethyl, propyl, isopropyl, butyl and pentyl).

R₃ is preferably an optionally substituted aryl group, e.g. a phenyl group, most preferably a 4-substituted phenyl group. The aryl group is preferably substituted by one or more —OR₅ groups, e.g. where R₅ is is alkyl (preferably 1-6 carbon atoms, eg methyl, ethyl, propyl, isopropyl, butyl or pentyl), alkynyl (preferably 2-7 carbon atoms) or optionally substituted aryl, eg where the substituents are selected from C₁-C₆-alkyl, C₁-C₆-alkoxy and halogen, such as chlorine.

R₁ and R₂ together with the carbon atoms to which they are attached preferably form a 5-10 membered heteroalkyl ring, eg having 1-3 heteroatoms selected from N, NR₄, O and S, most preferably a ring containing a single NR₄ group, e.g. a six membered piperidine ring. They preferably form a 3,3 di-substituted, 4,4-di-substituted, 1,3,3 tri-substituted or 1,4,4-tri-substituted piperidine.

Examples of R₄ are hydrogen, alkyl of 1-6 carbon atoms, —COR₅, —COOR₅, —SO₂R₃ and optionally substituted benzyl (eg 4-chlorobenzyl, 4-methoxybenzyl or 4-(2-piperidin-1-yl-ethoxy)benzyl).

Examples of R₅ are an optionally substituted alkyl of 1-18 carbon atoms (such as methyl, trifluoromethyl), an optionally substituted alkenyl of 2-18 carbon atoms, an optionally substituted aryl (such as phenyl), an optionally substituted 4-8 membered heteroaryl (such as pyridyl, thienyl) or an optionally substituted 5-10 membered cycloheteroalkyl (such as pyrrolyl); preferably R₅ is methyl, ethyl, n-butyl, t-butyl, but-2-ynyl, 4-chlorophenyl, 4-methoxyphenyl, 1-pyrrolidinyl, 3, pyridinyl, 2-thienyl, 2,2,5-trimethyl-1,3-dioxan-5-yl or 2-hydroxy-1-(hydroxymethyl)-1-methylethyl.

Typical optional substituents as used herein include C₁-C₆alkyl, C₁-C₆ alkoxy, C₁-C₆haloalkyl and halogen.

The present invention accordingly provides a pharmaceutical composition which comprises a compound of this invention in combination or association with a pharmaceutically acceptable carrier. In particular, the present invention provides a pharmaceutical composition which comprises an effective amount of compound of this invention and a pharmaceutically acceptable carrier.

The compositions are preferably adapted for oral administration. However, they may be adapted for other modes of administration, for example, parenteral administration for patients.

In order to obtain consistency of administration, it is preferred that a composition of the invention is in the form of a unit dose. Suitable unit dose forms include tablets, capsules, and powders in sachets or vials. Such unit dose forms may contain from 0.1 to 100 mg of a compound of the invention. The compounds of the present invention can be administered orally at a dose range of about 0.01 to 100 mg per kg. Such composition may be administered from 1 to 6 times a day, more usually from 1 to 4 times a day.

The compositions of the invention may be formulated with conventional excipients, such as fillers, a disintegrating agent, a binder, a lubricant, a flavoring agent, and the like. They are formulated in conventional manner.

### DETAILED DESCRIPTION OF THE INVENTION

Several synthetic routes can be employed to prepare the compounds of formula I, using alpha-sulfonylation of the enolisable carbonyl compound as the key step in the process. Several preferred routes for the preparation of these compounds are described in schemes I-III. Although, each sequence is illustrated with a compound of formula I, wherein X and Y are hydrogen, R₃ is aryl, and R₁ and R₂ taken together with the carbon atom to which they are attached form a 6-membered cycloheteroalkyl ring containing NR₄, additional compounds of this invention can be prepared in the same manner using the appropriate starting materials and routes as would be appreciated by one skilled in the art and illustrated by the specific examples. The reagents and the solvents for the individual step are given for illustrative purposes only and may be replaced by other reagents and solvents known to those skilled in the art.

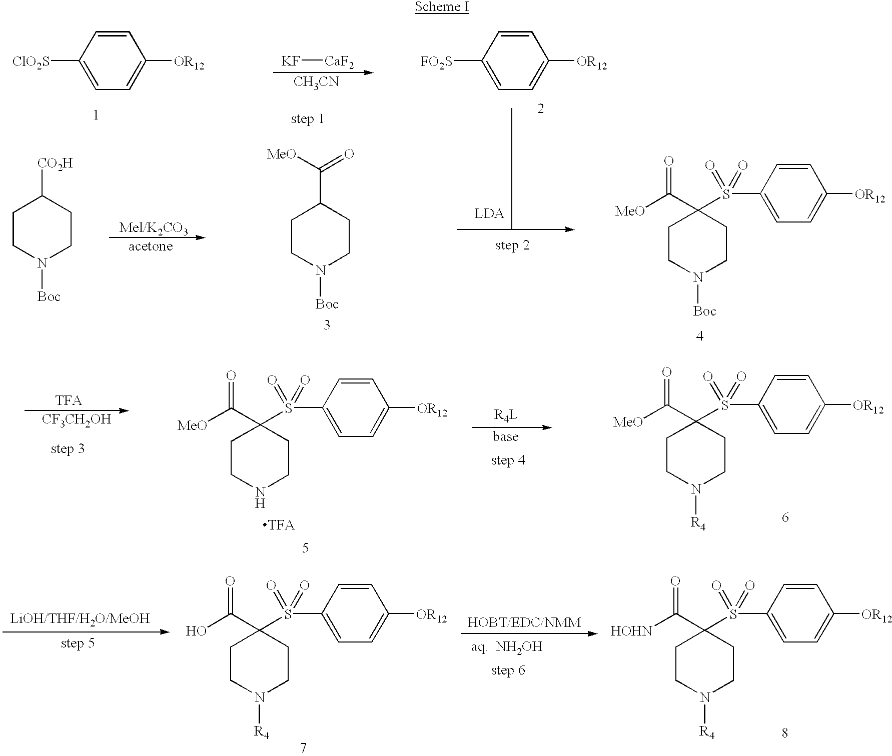

In Scheme I, step 1, sulfonyl chloride 1, wherein R₁₂ is methyl, n-butyl, 2-butynyl, or p-chlorophenyl, is treated with a potassium fluoride-calcium fluoride mixture (either commercially available or prepared according to the procedure by Ichihara) in acetonitrile at room temperature to obtain the sulfonyl fluoride 2.

In step 2, the enolate prepared from the ester 3 (prepared by treating commercially available Boc-isonipecotic acid with methyl iodide/potassium carbonate ) and lithium diisopropylamide (LDA) (prepared in situ using n-butyl lithium and diisopropylamine) is treated with compound 2 at −78° C.-25° C. to obtain the compound 4.

In step 3, the protecting group, t-butoxycarbonyl, is cleaved with trifluoroacetic acid in trifluoroethanol to obtain the compound 5 as a salt.

In step 4, R₄, as hereinabove defined, is introduced by treating compound 5 with R₄L, wherein L is a leaving group such as but not limited to halogen, in the presence of other reagents such as triethylamine and the solvents known to those skilled in the art, to obtain compound 6.

In step 5, the ester 6 is hydrolyzed with lithium hydroxide at 50° C. or sodium hydroxide for 15 hours to obtain acid 7.

In step 6, compound 7 is treated with N-hydroxybenzotriazole, 1-(3-dimethylaminopropyl)-3-ethylcarbodimide hydrochloride, N-methylmorpholine, and aqueous hydroxylamine to obtain the desired hydroxamic acid 8.

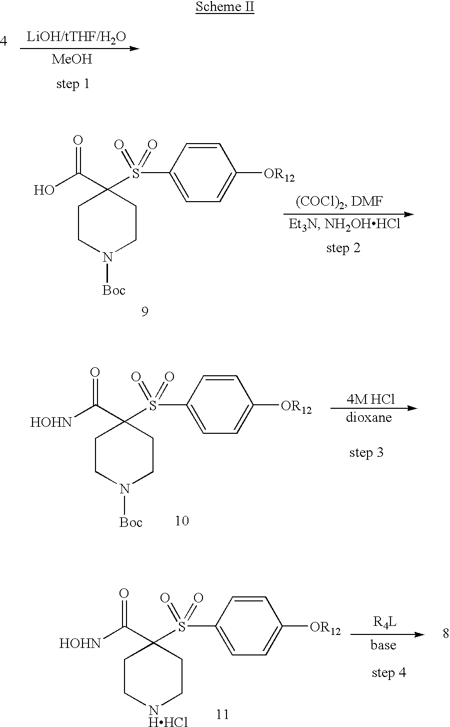

The target compounds can also be obtained by changing the order of transformations carried out for compound 4 as shown in the Scheme II.

In Scheme II, step 1, the compound 4 is treated with lithium hydroxide as in Scheme I, step 5, to obtain the N-protected carboxylic acid 9.

In step 2, the acid 9 is treated with oxalyl chloride, triethylamine, and hydroxylamine hydrochloride in dimethylformamide to obtain N-protected hydroxamic acid 10, which is then deprotected with 4M hydrochloric acid in dioxane to give the salt 11 in step 3.

In step 4, R₄ as hereinabove defined, is introduced selectively using the conditions in Scheme I, step 4 to obtain the hydroxamic acid 8.

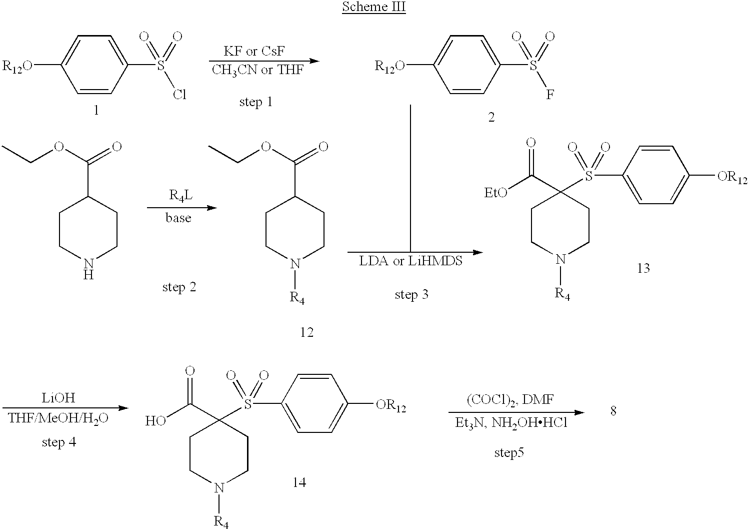

Alternatively, the target compounds can be obtained by following the synthetic sequence of Scheme III.

In Scheme III, step 1, sulfonyl fluoride 2 is obtained by treating sulfonyl chloride 1 with either potassium fluoride or cesium fluoride in acetonitrile. Alternatively, this reaction is carried out in tetrahydrofuran and the resulting solution is used for the next step without isolation of the sulfonyl fluoride.

In step 2, R₄ as hereinabove defined, is introduced early in the sequence by treating starting material such as ethyl isonipecotate R₄L, wherein L is a leaving group such as but not limited to halogen, in the presence of appropriate reagents such as triethylamine with commercially available ethyl isonipecotate.

In step 3, enolate prepared by reacting the compound 12 with lithium diisopropylamide or lithium hexamethyldisilazide, is treated with the fluoride 2 to obtain the compound 13.

In step 4 ester 13 is hydrolyzed with lithium hydroxide to give acid 14. Alternatively, step 3 and step 4 are carried out sequentially as a one-pot process without isolation of the ester 13.

In step 5, acid 14 is treated with oxalyl chloride, triethylamine, and hydroxylamine hydrochloride as in the Scheme II, step 2, to obtain the compound 8.

In the following examples, there are described several preferred embodiments to illustrate the invention. However, it should be understood that the invention is not intended to be limited to the specific embodiments.

General Procedure for the Preparation of Sulfonyl Fluorides from Sulfonyl Chlorides

Method A To a solution of the sulfonyl chloride (1 equiv) in acetonitrile was added potassium fluoride-calcium fluoride mixture (2 equiv with respect to potassium fluoride) and the resulting mixture was stirred for 4 hours at room temperature. The reaction mixture was filtered and the filtrate was concentrated. The crude product was dissolved in ethyl acetate and washed with water. The organic layer was dried over anhydrous sodium sulfate and the solvent was removed in vacuo to obtain the product.

Method B: To a solution of the sulfonyl chloride (1 equiv) in acetonitrile was added potassium fluoride (2 equiv). The resulting suspension was stirred for 18 hours at 20-25 ° C. The suspension was filtered and the solid was washed with diethylether. The mother liquor was concentrated in vacuo and resulting oil was seeded to give the product as a white crystalline solid.

Method C: To a solution of the sulfonyl chloride (1 equiv) in acetonitrile was added cesium fluoride (2 equiv). The resulting suspension was stirred for 18 hours at 20-25° C. The suspension was filtered and the solid washed with diethylether. The mother liquor was concentrated in vacuo and resulting oil was seeded to produce the product as a white crystalline solid.

Method D: The solution of sulfonyl chloride (1 equiv) in tetrahydrofuran was mixed with potassium fluoride (2 equiv) and stirred for 30 hours at 20-°C. The suspension was filtered and the solid was washed with tetrahydrofuran. This solution was used for the next step without isolation.

General Procedure for Alpha-sulfonylation of the Carbonyl Compound (Step 1)

To a solution of lithium diisopropylamide (1 equiv)(either commercially available or freshly prepared from n-butyllithium and diisopropylamine) in tertahydrofuran cooled to −78° C., was added a solution of the carbonyl compound (1 equiv) in tetrahydrofuran and the resulting mixture was stirred for 0.5-1 hour at that temperature. A solution of the sulfonyl fluoride (1.1 equiv) in tetrahydrofuran was then added to the mixture and the resulting mixture was stirred for 4-15 hours at room temperature, quenched with saturated aqueous ammonium chloride solution and extracted with ethyl acetate. The organic layer was washed with brine and dried over anhydrous sodium sulfate. The crude product was purified by either recrystallization or silica gel chromatography to obtain the desired product.

General Procedure for the Preparation of the Carboxylic Acid from the Ester (Step 3)

A solution of the ester (1 equiv) and lithium hydroxide or sodium hydroxide (1.5-2 equiv) in tetrahydrofuran/methanol/water (3:3:2) mixture was stirred at room temperature or heated at 55° C. for 15 hours. The mixture was concentrated, acidified to pH 3-5 with 1N aqueous hydrochloric acid, and extracted with ethyl acetate. The organic layer was washed with brine and dried over anhydrous sodium sulfate. Removal of the solvent under vacuuo gave the product.

General Procedure for the Preparation of Hydroxamic Acid from the Carboxylic Acid (Step 4)

Method A;

To a solution of the acid (1 equiv) in dimethylformamide was added hydroxybenzotriazol (1.2 equiv) followed by 1-(3-dimethylaminopropyl)-3-ethylcarbodimide hydrochloride (1.4 equiv) and N-methylmorpholine (1.5 equiv). The resulting mixture was stirred for 1 h at room temperature when 50% aqueous hydroxylamine solution (5 equiv) was added and the mixture was stirred for 15 h at that temperature. The solvent was removed in vacuo and ethyl acetate/water was added to the crude product. The organic layer was separated and washed successively with 1N aqueous hydrochloric acid, water, saturated aqueous sodium bicarbonate, and water. The organic layer was dried over anhydrous sodium sulfate and the solvent was removed in vacuo to obtain the product.

Method B;

To a solution of oxalyl chloride in methylene chloride was added dimethylformamide followed by the acid (1 equiv) in methylene chloride at 0° C. and the mixture was stirred for 1 hour at room temperature. This mixture was added to a solution containing hydroxylamine hydrochloride (10 equiv) and triethyl amine (15 equiv) in tetrahydrofuran/water (5:1) that had been stirring for 0.25-1 hour at 0° C. The reaction was allowed to warm to room temperature and stirred for 15-24 h at that temperature. The reaction mixture was concentrated and the residue was taken up in ethyl acetate. The organic layer was washed with saturated aqueous sodium bicarbonate and water and dried over anhydrous sodium sulfate. The solvent was removed in vacuo and the crude product was purified by triturating or silica gel chromatography to obtain the product.

### EXAMPLE 1

### 4-But-2-ynyloxybenzenesulfonyl fluoride

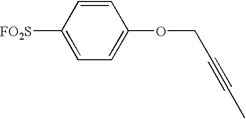

The general procedure for the preparation of sulfonyl fluorides was followed using 4-but-2-ynyloxybenzenesulfonyl chloride (2.0 g, 8.18 mmol) in acetonitrile (10 ml) and potassium fluoride-calcium fluoride mixture to obtain 1.5 g(80%) of the product as a solid.

IR: 2925, 2242, 1596, 1579, 1406, 1261, 997 cm⁻¹;

¹H NMR(300 MHz, CDCl₃):δ1.87(t, 3H, J=1.8 Hz), 4.76(q, 2H, J=1.8 Hz), 7.14(d, 2H, J=6.6 Hz), 7.95(d, 2H, J=6.6 Hz);

¹³C NMR(75 MHz, CDCl₃):δ3.6, 56.9, 72.4, 85.4, 115.8, 130.8, 163.3.

### EXAMPLE 2

### 1-(tert-Butyl)4-methyl 1,4-piperidinecarboxylate

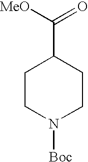

A mixture of N-t-butoxycarbonyl isonipecotic acid (20 g, 0.087 mmol), methyl iodide (62 g, 0.435 mmol), and potassium carbonate (120 g, 0.87 mmol) was stirred for 2 days. The mixture was filtered and the solvent was removed in vacuo. The crude product was dissolved in methylene chloride, washed with water and dried over anhydrous sodium sulfate. Removal of the solvent gave 20 g (95%) of the product as a white solid.

### EXAMPLE 3

### 4-(4-But-2-ynyloxybenzenesulfonyl)-piperidine-1,4-dicarboxylic acid tert-butyl ester methyl ester

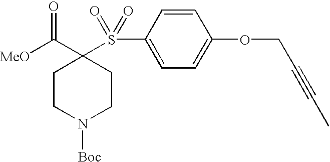

The general procedure for step 1 was followed using lithium diisopropylamide (70 mmol), product from Example 2 (15.5 g, 64 mmol), and the product from Example 1 (70 mmol) to obtain 24.5 g(85%) of the product as a white solid.

IR: 2978, 2242, 1740, 1697, 1594, 1418, 1301, 1002, 908cm⁻¹;

¹H NMR(300 MHz, CDCl₃):δ1.44(s, 9H), 1.87(m, 3H), 1.98(m, 2H), 2.32(m, 2H), 2.62(m, 2H), 3.74(s, 3H), 4.17(m, 2H), 4.74(m, 2H), 7.09(d, 2H, J=7.2 Hz) 7.71(d, 2H, J=7.2 Hz); ¹³C NMR(75 MHz, CDCl₃):δ4.0, 28.2, 28.7, 53.5, 57.2, 72.9, 73.1, 80.5, 85.4, 115.3, 127.0, 132.6, 154.7, 162.9, 167.8;

HR-MS: Calculated for C₂₂H₂₉NO₇S (M+Na) 474.1557; Found 474.1547.

### EXAMPLE 4

### 1-(tert-Butoxycarbonyl)-4-{[4-(2-butynyloxy)phenyl]sulfonyl}-4-piperidinecarboxylic acid

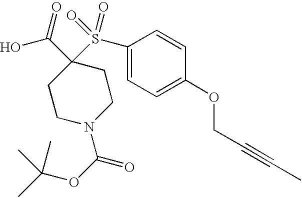

The general procedure for step 3 was followed using the product from Example 3 (15 g, 33.2 mmol) in water (100 ml), methanol (50 ml), tetrahydrofuran (50 ml) and lithium hydroxide hydrate (2.73 g, 66.4 mmol) at reflux temperature for 8 hours to obtain 14.5 g (100%) of the acid as a white powder.

¹H NMR(300 MHz, DMSO-d₆):δ1.38 (s, 9H), 1.7-1.8 (m, 2H), 1.85 (t, 3H, J=2.2 Hz), 2.2-2.3 (m, 2H), 2.5-2.7 (m, 2H), 3.95-4.05 (m, 2H), 4.89 (q, 2H, J=2.2 Hz), 7.1-7.8 (m, 4H); MS-ES: m/z 482 (M−H);

Analysis for C₂₁H₂₇NO₇S): Calculated: C, 57.65; H, 6.22; N, 3.20; Found: C, 57.59; H, 6.49; N, 3.20.

### EXAMPLE 5

### tert-Butyl 4-{[4-(2-butynyloxy)phenyl]sulfonyl}-4-[(hydroxyamino)carbonyl]-1-piperidinecarboxalate

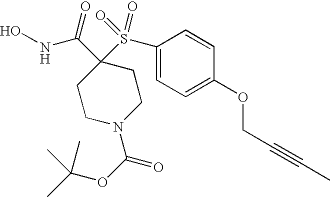

The general procedure for step 4 was followed using dimethylformamide (3.53 ml, 46 mmol), oxalyl chloride (22.9 ml of a 2.0M solution in dichloromethane), the product from Example 4 (10 g, 22.9 mmol), hydroxylamine hydrochloride (16 g, 229 mmol), and triethylamine (48 m, 344 mmol) to obtain the product as a white powder 6.3 g (61%).

¹H NMR(300 MHz, DMSO-d₆):δ1.38 (s, 9H), 1.6-1.7 (m, 2H), 1.85 (t, 3H, J=2.2 Hz), 2.2-2.3 (m, 2H), 2.5-2.7 (m, 2H), 3.9-4.0 (m, 2H), 4.87 (q, 2H, J=2.2 Hz), 7.1-7.7 (m, 4H);

MS-ES: m/z 453 (M+H)⁺.

### EXAMPLE 6

### 4-{[4-(2-Butynyloxy)phenyl]sulfonyl}-N-hydroxy-4-piperidinecarboxamide hydochloride

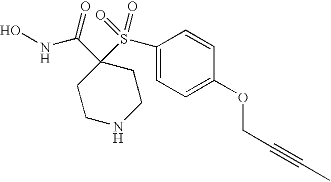

To a solution of product from Example 5 (6.3 g, 13.9 mmol) in methylene chloride was added 4N hydrochloric acid in dioxane. After 6 hours the reaction mixture was concentrated in vacuo. Methanol was added and the resulting mixture was concentrated in vacuo. Methylene chloride was added and removed in vacuo (2×). Trituration with diethyl ether gave the product as a white powder 5.14 g.

¹H NMR(300 MHz, DMSO-d₆):δ1.86 (t, 3H, J=2.2 Hz), 2.0-2.7 (m, 8H), 4.89 (q, 2H, J=2.2 Hz), 7.1-7.8 (m, 4H), 8.8-11.0 (m, 4H);

MS-ES: m/z 353 (M+H)⁺.

### EXAMPLE 7

### 4-(4-Chlorophenoxy)phenylsulfonyl fluoride

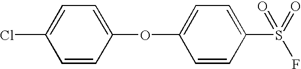

The general procedure for the preparation of sulfonyl fluorides was followed using 4-(4-Chlorophenoxy)phenylsulfonyl fluoride (770 mg, 2.54 mmol) and potassium fluoride-calcium fluoride mixture (1.47 g, 2 equiv) to obtain 660 mg (91%) of the product.

IR: 1599, 1579, 1484, 1395, 1258, 1210, 1183, 768 cm⁻¹; ¹H NMR(300 MHz, CDCl₃):δ7.03-7.13 (m, 4H), 7.38-7.43 (m, 2H), 7.93-8.00 (m, 2H);

¹³C NMR(75 MHz, CDCl₃):δ117.6, 117.7, 122.0, 129.7, 130.5, 131.1, 152.9, 163.7;

MS-ES: m/z 285.9.

### EXAMPLE 8

### 1-(tert-Butyl) 4-methyl 4-{[4-(4-chlorophenoxy)phenyl]sulfonyl}-1,4-piperidinedicarboxylate

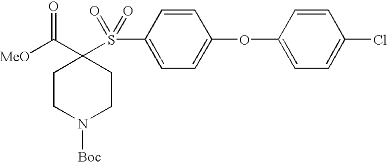

The general procedure for step 1 was followed using lithium diisopropylamide (2.31 mmol), the product from Example 1 (510 mg, 2.1 mmol), and the product from Example 7 (600 mg, 2.2 mmol) to obtain 520 mg (49%) of the product as a solid.

IR: 1727, 1682, 1485, 1427, 1252, 1153 cm⁻¹;

¹H NMR(300 MHz, CDCl₃):δ1.44 (s, 9H), 1.97-2.07 (m, 2H), 2.29-2.33 (m, 2H), 2.62 (br s, 2H), 3.76 (s, 3H), 4.08-4.15 (m, 2H), 7.01-7.07 (m, 4H), 7.36-7.42 (m, 2H), 7.68-7.73 (m, 2H);

¹³C NMR(75 MHz, CDCl₃):δ28.3, 53.3, 72.6, 80.2, 117.1, 121.9, 128.5, 130.4, 130.7, 132.6, 153.2, 154.4, 162.8, 167.4;

HR-MS: m/z Calculated for C₂₄H₂₈ClNO₇S (M+Na) 532.1167; Found 532.1152.

### EXAMPLE 9

### 1-(tert-Butoxycarbonyl)-4-{[4-(4-chlorophenoxy)phenyl]sulfonyl}-4-piperidine carboxylic acid

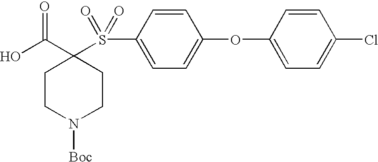

The general procedure for step 3 was followed using the product from Example 8 (450 mg, 0.88 mmol) and lithium hydroxide (32 mg, 1.32 mmol) in tetrahydrofuran (3 ml)/methanol (3 ml)/water (2 ml) at 55° C. for 15 hours to obtain 375 mg (86%) of the product.

IR: 3438, 2976, 1693, 1627, 1484, 1248, 1139 cm⁻¹;

¹H NMR(300 MHz, DMSO-d₆):δ1.38 (s, 9H), 1.55-1.64 (m, 2H), 2.09 (s, 1H), 2.13 (s, 1H), 2.68 (br s, 2H), 3.39 (br s, 1H), 3.90 (m, 2H), 7.06 (d, 2H, J=9.0 Hz), 7.16 (d, 2H, J=12.0 Hz), 7.52 (d, 2H, J=12.0 Hz), 7.70 (d, 2H, J=9.0 Hz);

¹³C NMR(75 MHz, DMSO-d₆):δ116.6, 121.9, 128.8, 130.2, 131.2, 132,7, 153.7, 153.8, 160.7, 165.2;

HR-MS: m/z Calculated for C₂₃H₂₆ClNO₇S(2M+H) 991.2311; Found 991.2273.

### EXAMPLE 10

### 1-(tert-Butyl)-4-{[4-(4-chlorophenoxy)phenyl]sulfonyl}-4-[(hydroxyamino)carbonyl]-1-piperidinecarboxylate

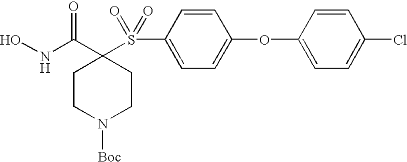

The general procedure for step 4 was followed using the product from Example 9 (350 mg, 0.71 mmol) in dimethylformamide (7 ml), hydroxybenzotriazol (114 mg, 0.85 mmol), 1-(3-dimethylaminopropyl)-3-ethylcarbodimide hydrochloride (190 mg, 0.99 mmol), N-methylmorpholine (117 uL, 1.06 mmol), and 50% aqueous hydroxylamine (217 uL, 3.55 mmol) to obtain 150 mg (41%) of the product.

IR: 3739, 3382, 2931, 1664, 1484, 1249, 1150 cm⁻¹.

¹H NMR(300 MHz, DMSO-d₆):δ1.38 (s, 9H), 1.68 (m, 2H), 2.14 (m, 2H), 2.51 (m, 2H), 3.95 (m, 2H), 7.14 (d, 2H, J=9 Hz), 7.20 (d, 2H, J=9 Hz), 7.55 (d, 2H, J=9 Hz), 8.01 (d, 2H, J=9 Hz), 9.20 (s, 1H), 11.02 (s, 1H);

¹³C NMR(75 MHz, DMSO-d₆):δ27.9, 70.0, 79.2, 117.3, 122.2, 128.4, 129.2, 130.4, 132.8, 153.3, 153.7, 160.2, 161.8;

HR-MS:m/z Calculated for C₂₃H₂₇ClN₂O₇S(2M+H) 1021.2527; Fund 1021.2523.

### EXAMPLE 11

### 4-{[4-(4-Chlorophenoxy)phenyl]sulfonyl}-N-hydroxy-4-piperidinecarboxamide

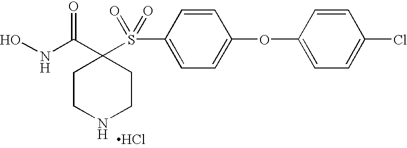

To a solution of product from Example 10 (105 mg, 0.21 mmol) in methylene chloride (20 ml) was added a 4M hydrochloric acid solution (258 μL, 1.03 mmol) and the resulting mixture was stirred for 4 hours at room temperature. The solvent was removed and diethyl ether was added. The precipitated solid was filtered and dried to obtain 80 mg (85%) of the product.

IR: 3392, 3214, 2875, 1664, 1484, 1250, 1142, 1087 cm⁻¹;

¹H NMR(300 MHz, DMSO):δ2.13 (m, 2H), 2.46 (m, 2H), 2.59 (m, 2H), 3.33 (m, 2H), 7.19 (m, 4H), 7.52 (d, 2H, J=9.0 Hz), 7.72 (d, 2H, J=9.0 Hz), 9.19 (br s, 1H), 9.56 (br s, 1H);

HR-MS: m/z Calculated for C₁₈H₁₉ClN₂O₅S(M+H)) 411.0776; Found 411.0777.

### EXAMPLE 12

### Piperidine-1,3-dicarboxylic acid 1-tert-butyl 3-ethyl ester

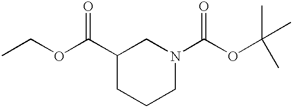

To a stirred solution of ethyl nipecotate (5.1 g, 33 mmol) in methylene chloride (75 ml) and triethylamine (3.7 g, 36 mmol) was added in portions di-t-butyl-dicarbonate (7.1 g, 33 mmol). The reaction mixture was stirred at room temperature for 18 hours, quenched with ice water and extracted with chloroform. The organic layer was dried over sodium sulfate, filtered, concentrated and chromatographed on a silica-gel column with 20:80 ethyl acetate:hexane. Piperidine 1,3dicarboxylic acid 1-tert-butyl ester-3-ethyl ester was isolated as a waxy solid, 6.86 g (82%).

¹H NMR(300 MHz, CDCl₃):δ1.26 (t, 3H), 1.46 (s, 9H), 1.63 (m, 2H), 2.03 (m, 1H), 2.41 (m, 1H), 2.76 (m, 2H), 3.89 (m, 1H), 4.14 (m, 2H);

MS-ES:m/z 258.2 (M+H)⁺;

Analalysis for C₁₃H₂₃NO₄, Calculated: C, 60.68; H, 9.08; N, 5.44 Found: C, 60.60; H, 9.10; N, 5.38.

### EXAMPLE 13

### 4-Methoxyphenylsulfonyl fluoride

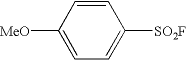

The general procedure for the preparation of sulfonyl fluorides was followed using 4-methoxyphenylsulfonyl chloride (11.0 g, 53 mmol) and potassium fluoride-calcium fluoride mixture (17.0 g) in acetonitrile (100 ml) to obtain 10.0 g(100%) of the product.

MS-ES: m/z 187.0(M−H)⁻.

### EXAMPLE 14

### 1-(tert-Butyl)-3-ethyl-3-[(4-methoxyphenyl)sulfonyl]-1,3-piperidine dicarboxylate

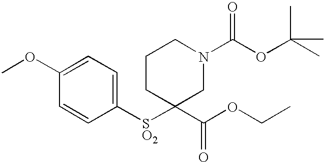

The general procedure for step 1 was followed using lithium diisopropylamide (28 mmol), product from Example 12 (5.3 g, 28 mmol), and product from Example 13 (5.3 g, 28 mmol) to obtain 7.2 g (60%) of the product.

¹H NMR(300 MHz, DMSO-d₆):δ1.15 (t, 3H), 1.44 (s, 9H), 1.69 (m, 2H), 2.14 (m, 2H), 3.17 (m, 2H), 3.35 (d, 2H), 3.8 (s, 3H), 4.06 (m, 2H), 7.19 (d, 2H), 7.69 (d, 2H);

MS-ES: m/z 428.5 (M+H)⁺;

Analysis for C₂₀H₂₉NO₇S Calculated: C, 56.19; H, 6.84; N, 3.28 Found: C, 56.84; H, 7.20; N, 3.48.

### EXAMPLE 15

### Ethyl 3-[(4-methoxyphenyl)sulfonyl]-3-piperidinecarboxylate

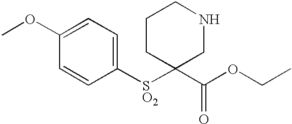

To a stirred solution of product from Example 14 (1.72 g, 4.0 mmol) in methylene chloride(25 ml) at 0° C. was added a saturated solution of hydrogen chloride in methylene chloride (25 ml). After 5 hours the solution was concentrated to afford 1.23 g (84.5%) of the product.

MS-ES: m/z 328.3 (M+H)⁺;

Analalysis for C₁₅H₂₁NO₅S Calculated: C, 49.51; H, 6.09; N, 3.85 Found: C, 47.91; H, 7.08; N, 4.16;

¹H NMR(300 MHz, DMSO-d₆):δ1.09 (t, 3H), 2.29 (d, 2H), 2.99 (m, 2H), 3.07 (m, 2H), 3.72 (d, 2H), 3.89 (s, 3H), 4.11 (m, 4H), 7.22 (d, 2H), 7.72 (d, 2H).

### EXAMPLE 16

### Ethyl 1-benzyl-3-[(4-methoxy)sulfonyl]-3-piperidinecarboxylate

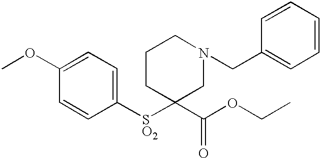

A solution of product from Example 15 (1.23 g, 3.4 mmol), benzyl bromide (0.64 g, 3.7 mmol) and dry powdered potassium carbonate (3.8 g) in dry acetone (60 ml) was heated at reflux temperature for 18 hours. The mixture was cooled and the potassium salts were removed by filtration and the filtrated was concentrated. The residue was dissolved in chloroform, washed with water, dried over sodium sulfate and concentrated to afford 1.8 g (94%) of the product as a yellow oil.

¹H NMR(300 MHz, DMSO-d₆):δ1.04 (t, 3H), 2.71 (m, 2H), 3.39 (m, 3H), 3.54 (m, 2H), 3.38 (m, 4H), 3.92 (s, 3H), 4.02 (m, 4H), 4.54 (s, 2H), 7.13 (d, 2H), 7.21 (d, 2H), 7.29 (d, 2H), 7.62 (d, 2H);

MS-ES: m/z418.5 (M+H)⁺.

### EXAMPLE 17

### 1-Benzyl-3-(4-methoxybenzenesulfonyl)-piperidine-3-carboxylic acid

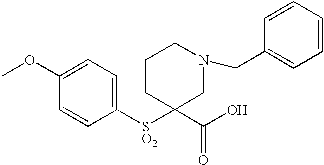

The general procedure for step 3 was followed using the product from Example 16 (1.7 g, 4.0 mmol), sodium hydroxide (10N, 3 ml), methanol (10 ml) and tetrahydrofuran (10 ml) at 50° C. for 2 hours to obtain 1.13 g (67%) of the product, mp 103° C.

¹H NMR(300 MHz, DMSO-D₆):δ1.04 (t, 3H), 2.71 (m, 2H), 3.36 (m, 6H), 3.55 (m, 2H), 3.85 (3, 3H), 7.12 (d, 2H), 7.27 (d, 2H), 7.64 (d, 2H), 7.77 (d, 2H);

MS-ES: n/z 344.4 (M−H) —CO₂.

### EXAMPLE 18

### 1-Benzyl-3-(4-methoxybenzenesulfonyl)piperidine-3-carboxylic acid hydroxamide

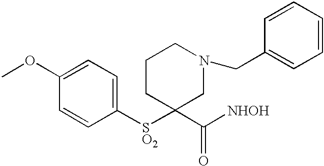

To a stirred solution of product from Example 17 (1 .g, 2.9 mmol) and dimethylformamide (5 ml) in methylene chloride (30 ml) at 0° C. was added, dropwise, oxalyl chloride (1.8 gm, 14.5 mmol). After the addition, the reaction mixture was stirred at room temperature for 1 hour. Simultaneously, in a separate flask a mixture of hydroxyl-amine hydrochloride (1.6 gm, 23 mmol) and triethylamine (3 ml, excess) was stirred in tetrahydrofuran:water (5:1, 30 ml) at 0° C. for 1 hour. At the end of 1 hour, the oxalyl chloride reaction mixture was concentrated and the pale yellow residue was dissolved in 10 ml of methylene chloride and added slowly to the hydroxylamine solution at 0° C. The reaction mixture was stirred at room temperature for 24 hours and concentrated. The residue obtained was extracted with chloroform and washed well with water. The product obtained was purified by silica gel column chromatography; eluted with 2% methanol:chloroform. The product was converted to the hydrochloride salt by dissolving in methanol (10 ml) at 5° C. and adding saturated hydrogen chloride in methanol (5 ml). 1-Benzyl-3-(4-methoxy-benzenesulfonyl)-piperidine-3-carboxylic acid hydroxamide propionamide was isolated as a white solid, 1.17 g, (91%), mp 132.9° C.

¹H NMR(300 MHz, DMSO-d₆):δ1.08 (m, 23H), 2.49 (m, 2H), 3.87(s, 3H), 4.25 (d, 2H), 7.10 (d, 2H), 7.44 (s, 5H), 7.58 (d, 2H), 8.85 (s, 1H), 9.45 (s, 1H);

MS-ES: m/z 405.3(M+H)⁺.

### EXAMPLE 19

### 1-(tert-Butyl) 4-ethyl 4-{[4-2-butynyloxy)phenyl]sulfonyl}-1,4-piperidine dicarboxylate

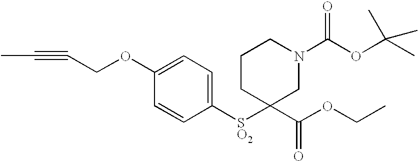

The general procedure for the preparation of sulfonyl fluorides was followed using lithium diisopropylamide (20 mmol), product from Example 1 (4.4 g, 19.5 mmol), and product from Example 12 (5.0 g, 19.5 mmol) to obtain 10.97 g (76%) of the product, mp 103.4° C.

¹H NMR (300 MHz, DMSO-d₆):δ1.07 (t, 3H), 1.34 (s, 9H), 3.31 (s, 3H), 3.84 (m, 2H), 4.00 (m, 4H), 4.53 (d, 2H), 4.91 (m, 4H), 7.22 (d, 2H), 7.71 (d, 2H);

MS-ES: m/z 466.4(M+H)⁺;

Analysis for C₂₃H₃₁ClNO₇S) Calcuclated: C, 59.34, H, 6.71; N, 3.01 Found: C, 59.49; H, 6.84; N, 3.16.

### EXAMPLE 20

### Ethyl 3-{[4-(2-butynyloxy)phenyl]sulfonyl}-3-piperidinecarboxylate

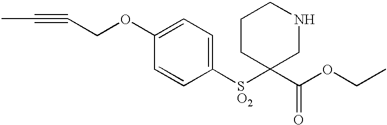

Following the procedure of Example 15, using the product from Example 19 (5.45 g, 11.7 mmol) in dissolved in methylene chloride, the desired product was obtained as a white solid 3.47 g (74%). The solid is very hydroscopic and is store under nitrogen.

¹H NMR(300 MHz, DMSO-d₆):δ1.08 (t, 3H), 2.30 (bd, 1H), 2.96 (t, 2H), 3.07 (m, 2H), 3.33 (s, 3H), 3.38 (m, 4H), 4.09 (m, 2H), 4.93 (s, 2H), 7.26 (d, 2H), 7.74 (d, 2H);

MS-ES:m/z 366.2 (M+H)⁺;

Analysis for C₁₈H₂₃O₅S Calculated: C, 53.79; H, 6.02; N, 3.49 Found: C, 52.34; H, 6.17; N, 3.52.

### EXAMPLE 21

### Ethyl 3-{[4-(2-butynyloxy)phenyl]sulfonyl}-1-ethyl-3-piperidinecarboxylate

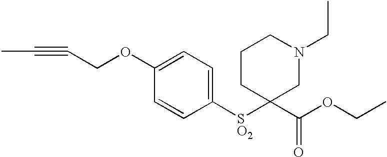

Following the procedure of Example 16, using the product from Example 20 (2.97 g, 8.0 mmol) in dry acetone (50 ml), the desired product was isolated as an amber gum, 3.47 g (99%).

¹H NMR(300 MHz, DMSO-d₆):δ0.89 (t, 3H), 1.05 (t, 3H), 2.72 (d 2H), 3.28 (m, 2H), 3.31 (s, 3H), 4.01 (m, 4H), 4.91 (m, 2H), 7.19 (d, 2H), 7.70 (d, 2H);

MS-ES: m/z 394.3 (M+H)⁺.

### EXAMPLE 22

### 3-{[4-(2-Butynyloxy)phenyl]sulfonyl}-1-ethyl-3-piperidinecarboxylic acid

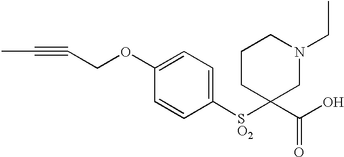

The general procedure for step 3 was followed using the product from Example 21 (3.2 g, 8.0 mmol) in tetrahydrofuran:methanol (15:25 ml), and sodium hydroxide (15 ml) at 50° C. for 2 hours to obtain 2.11 g (71%) of the product as a white solid: mp 159.2° C.

¹H NMR (300 MHz, DMSO-d₆):δ1.02 (t, 3H), 2.70 (m, 4H), 2.92 (d, 2H), 3.47 (d, 2H), 4.865 (m, 2H), 7.09 (d, 1H), 7.17 (d, 1H), 7.60 (d, 1H), 7.68 (d, 1H);

MS-ES: m/z 366.3 (M+H)⁺;

Analysis for C₁₈H₂₃O₅ Calculated: C, 59.16; H, 6.34; N, 3.83 Found: C, 59.2; H, 6.45; N, 3.67.

### EXAMPLE 23

### 3-{[4-(2-Butynyloxy)phenyl]sulfonyl}-1-ethyl-N-hydroxy-3-piperidinecarboxamide

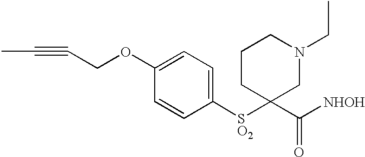

Following the procedure of Example 18, using the product from Example 22 (2.0 g, 5.5 mmol), 0.193 g (10%) of the desired product was isolated as a white solid, mp190° C.

¹H NMR(300 MHz, DMSO-d₆):δ1.18 (m, 3H), 1.97 (m, 2H), 2.55 (m, 2H), 3.21(m, 5H), 3.52 9S, 3H), 3.82 (d, 1H), 4.91 (m, 2H), 7.19 (d, 2H), 7.51 (s, 5H), 8.67 (s, 1H), 9.48 (s, 1H); MS-ES: m/z 405.3 (M+H)⁺.

Analysis for C₁₈H₂₄N₂O₅S Calculated: C, 51.86; H, 6.04; N, 6.72 Found: C, 50.03; H, 6.33; N, 6.42.

### EXAMPLE 24

### Ethyl 3-{[4-(2-butynyloxy)phenyl]sulfonyl}-1-(4-chlorobenzyl)-3-piperidinecarboxylate

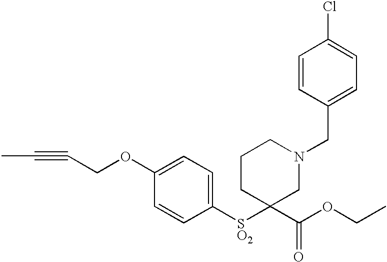

Following the procedure of Example 16, using the product from Example 20 (2.97 g, 8.0 mmol) in dry acetone (50 ml), 1.66 g (99%) of the product was isolated as a brown oil.

MS-ES: m/z 491.3 (M+H)⁺.

### EXAMPLE 25

### 3-{[4-(2-Butynyloxy)phenyl]sulfonyl}-1-(4-chlorobenzyl)-3-piperidinecarboxylic acid

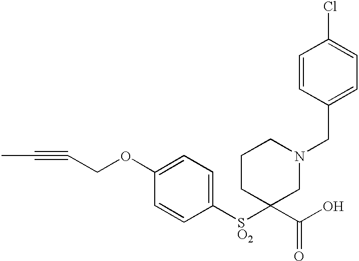

The general procedure for step 3 was followed using the product from Example 24 (1.64 g, 8.0 mmol) in tetrahydrofuran:methanol (15:50 ml) and sodium hydroxide (15 ml) at 50° C. for 2 hours to obtain 1.11 g (75%) of the product as a white solid, mp 115.2° C.

¹H NMR(300 MHz, DMSO-d₆):δ2.33 (d, 2H), 2.7 (d, 2H), 3.29 (s, 32H), 3.33 9 m, 2H), 3.52 (q, 2H), 4.47 (s, 2H), 4.81 (s, 2H), 7.16 (d, 2H), 7.27 (d, 2H), 7.34 (d, 2H), 7.67 (d, 2H);

MS-ES: m/z 462.1 (M+H)⁺;

Analysis for C₂₃H₂₄ClNO₅S Calculated: C, 59.16; H, 6.34; N, 3.83 Found: C, 59.64; H, 5.65; N, 2.66.

### EXAMPLE 26

### 3-{[4-(2-Butynyloxy)phenyl]sulfonyl}-1-(4-chlorobenzyl)-N-hydroxy-3-piperidinecarboxamide

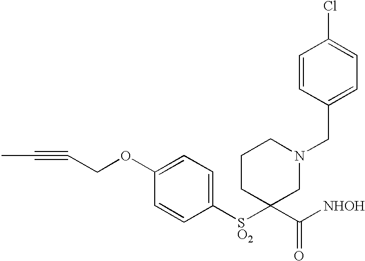

Following the procedure of Example 18, using the product from Example 25 (2.0 g, 5.5 mmol), 0.48 g (43%) of the product was isolated as a white solid, mp 124.4° C.

¹H NMR(300 MHz, DMSO-d₆):δ2.0 (m, 2H), 339 (m, 5H), 4.27 (d, 2H), 4.89 (m, 2H), 7.14 (d, 42H), 7.15 (m, 45H), 7.61 (d, 2H), 8.95 (s, 1H), 9.46 (s, 1H);

MS-ES: m/z 477.1 (M+H)⁺;

Analysis for C₁₈H₂₄N₂O₅S Calculated: C, 53.8; H, 5.10; N, 5.46 Found: C, 51.4; H, 5.42; N, 6.32.

### EXAMPLE 27

### Sodium-4-butoxybenzenesulfonic acid

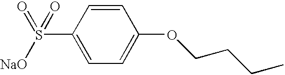

To a suspension of sodium-4-hydroxybenzenesulfonic acid (40.0 g, 0.172 mol) in 2-propanol (300 ml) was added 1N sodium hydroxide (190 ml, 0.189 mol). After 10 minutes n-butylbromide (38.9 g, 0.28 mol) was added and the hazy solution was heated at reflux temperature. The reaction mixture was partially evaporated, filtered, washed with diethyl ether, and dried to give 38.6 g (88.4%) of the product.

¹H NMR(300 MHz, DMSO-d₆):δ7.5 (d, 2H, J=8.7 Hz), 6.83 (d, 2H, J=8.7 Hz), 3.96 (t, 2H, J=6.5 Hz), 1.68 (m, 2H), 1.42 (m, 2H), 0.92 (t, 3H, J=7.4 Hz);

HPLC: 99.94% area; LC-MS: consistent.

### EXAMPLE 28

### 4-n-Butoxybenzenesulfonyl chloride

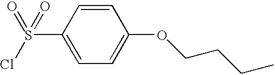

To the product from Example 27 (34.0 g, 0.134 mol) was added phosphorous oxychloride (60 ml, 0.643 mol) and the heterogeneous mixture was heated at reflux temperature (105° C.) for 4 hours. After 4 hours the reaction mixture was cooled to ambient temperature and ice water (600 ml) was added while stirring. The mixture was extracted with diethyl ether. The organic layer was washed with water (200 ml), saturated sodium bicarbonate solution (200 ml) and water (200 ml). The organic layer was dried over anhydrous sodium sulfate and the solvent was removed to give 33.3 g (99.3%) of the product as a colorless liquid.

¹H NMR(300 MHz, CDCl3):δ7.96 (d, 2H, J=6.0 Hz), 7.02 (d, 2H, J=6.0 Hz), 4.07 (t, 2H, J=4.3 Hz), 1.82 (m, 2H), 1.51 (m, 2H), 0.99 (t, 3H, J=4.9 Hz);

GC-MS: 100% pure.

### EXAMPLE 29

### 4-n-Butoxybenzenesulfonyl fluoride

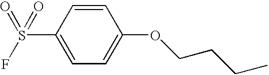

To a solution of product from Example 28 (33.3 g, 0.134 mol) in acetonitrile (200 ml) was added potassium fluoride on calcium fluoride (85.8 g, 0.298 mol) and the resulting heterogeneous mixture was stirred at ambient temperature for 20 hours. The reaction mixture was filtered, washed with acetonitrile (20 ml×2) and evaporated. The oily residue was dissolved in ethyl acetate (200 ml), washed with saturated sodium chloride solution (200 ml), dried over anhydrous sodium sulfate and concentrated to give 30.4 g, (98%) of the product as a clear colorless liquid.

¹H NMR(300 MHz, CDCl3):δ7.92 (d, 2H, J=6.0 Hz), 7.04 (d, 2H, J=6.0 Hz), 4.06 (t, 2H, J=4.3 Hz), 1.82 (m, 2H), 1.5 (m, 2H), 0.99 (t, 3H, J=4.9 Hz);

GC-MS: 97.6% pure, 2.4% starting material.

### EXAMPLE 30

### 1-[4-(2-Piperidin-1-yl-ethoxy)benzyl]piperidine-4-carboxylic acid methyl ester

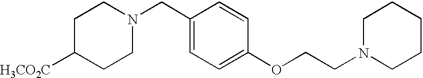

A mixture of methyl isonipecotate (5.0 g, 34.9 mmol), 4-piperidine ethoxy benzyl chloride hydrochloride (10.13 g, 34.9 mmol) and potassium carbonate (10.6 g, 76.6 mmol, −325 mesh) in acetone was heated at reflux temperature for 24 hours. After cooling to room temperature, the reaction was filtered, washed with acetone (25 ml×3), and evaporated to give a light brown oil. The oil was dissolved in ethyl acetate (100 ml), washed with water (100 ml×2), saturated sodium chloride solution (100 ml), dried over sodium sulfate and concentrated to afford 9.0 g (72%) of the product as a light brown oil.

¹H NMR(300 MHz, CDCl3):δ7.19 (d, 2H, J=5.7 Hz), 6.84 (d, 2H, J=5.7 Hz), 4.09 (t, 2H, J=4.06 Hz), 3.66 (s, 3H), 3.41 (s, 2H), 2.8 (m, 4H), 2.51 (m, 4H), 2.3 (m, 1H), 2.02 (m, 2H), 1.87 (m, 2H), 1.76 (m, 2H), 1.51 (m, 4H), 1.45 (m, 2H);

GC-MS: 94.1% pure.

### EXAMPLE 31

### 4-(4-Butoxybenzenesulfonyl)-1-[4-(2-piperidin-1-yl-ethoxy)benzyl]piperidine-4-carboxylic acid methyl ester

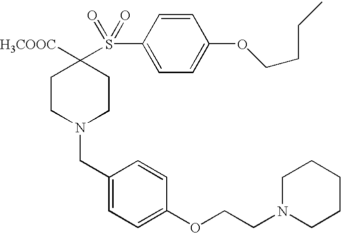

To a 0° C. solution of diisopropylamine (0.67 ml, 4.8 mmol) in tetrahydrofuran (6 ml) at was added n-butyllithium (2.0 ml, 2.5M in hexane). The resulting mixture was stirred for 20 minutes, cooled to −78° C. and a solution of product from Example 30 (1.5 g, 4.16 mmol) in tetrahydrofuran (6 ml)was added dropwise. After 1 hour at −78° C., the product from Example 3(1.01 g, 4.36 mmol) in tetrahydrofuran (4 ml) was added in one portion and the mixture warmed to ambient temperature. After 3 hours, the reaction mixture was quenched with saturated ammonium chloride solution (8 ml)and extracted with ethyl acetate (20 ml×2). The organic layer was washed with water (30 ml) and saturated sodium chloride solution (30 ml), dried over sodium sulfate and concentrated to give 2.24 g of the crude product as a brown syrup.

MS-ES: m/z 573 (M+H)⁺.

### EXAMPLE 32A

### 1-Benzylpiperidine-4-carboxylic acid ethyl ester

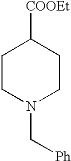

To a solution of ethyl isonipecotate (72.8 g, 0.45 mol) in ethanol (150 ml) was added benzyl bromide (101 g, 0.59 mol), dropwise, at 0 to 10° C., followed by triethylamine (68.8 g, 0.68 mol). The resulting suspension was warmed to ambient temperature and stirred for 6 hours. The reaction mixture was diluted with water (200 ml) and extracted with ethyl acetate (3×150 ml). The organic layer was dried over anhydrous magnesium sulfate, filtered through silica pad, and concentrated to afford 98.6 g (89%) of the product as a yellow viscous liquid.

¹H-NMR(CDCl₃):δ7.9-7.0 (m, 5H), 4.4-4.1(q, 2H), 3.5 (s,2H), 2.9-2.8 (m, 4H), 2.6-2.3 (m, 1H), 2.1-1.6 (m, 4H), 1.3-1.2 (t, 3H);

GC-MS: 99.4% pure.

### EXAMPLE 32B

### 1-Benzylpiperidine-4-carboxylic acid methyl ester

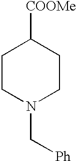

The above named compound was prepared from methyl isonipecotate in methanol using the procedure of Example 32A (yield 97%);

¹H-NMR(CDCl₃):δ7.7-6.9 (m, 5H), 3.7 (s, 3H), 3.5 (s, 2H), 3.0-2.8 (m, 4H), 2.4-2.2 (m, 1H), 2.1-1.6 (m, 4H);

GC-MS: 92% pure.

### EXAMPLE 33

### 4-[4-(4-Chlorophenoxy)benzenesulfonyl]-1-benzylpiperidine-4-carboxylic acid methyl ester

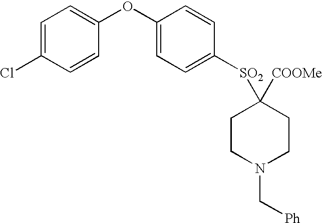

Prepared with LDA as Base:

Freshly distilled diisopropylamine (1.58 g, 15.6 mmol) was dissolved in tetrahydrofuran (18 ml) and cooled to 0° C. A solution of 2.5M n-butyl lithiumi in hexane (5 ml, 12.5 mmol) was added at a temperature below 5° C. and the resulting yellow solution was stirred for 0.5 hour while cooling to −20° C. A solution of product from Example 32B (1.46 g, 6.25 mmol) in tetrahydrofuran (5 ml) was added, dropwise, at −20° C. and the resulting mixture was stirred for 2 hours. A solution of product from Example 6 in tetrahydrofuran (5 ml) was added at −20 to −25° C. and the dark yellow reaction mixture was stirred for 1 hour at −20° C. The mixture was quenched with saturated ammonium chloride (20 ml) and extracted with ethyl acetate (3×15 ml). The organic solution was dried with magnesium sulfate, filtered through silica pad and concentrated to a small residual volume. The residue was triturated with isopropyl ether (10 ml) to produce 1.73 g (69%) of the product as yellow gummy crystals.

¹H-NMR(CDCl₃):δ7.8-7.0 (m, 13H), 3.7 (s, 3H), 3.4 (s, 2H), 3.0-1.8 (m, 8H); HPLC: 87% pure.

Prepared with LiHMDS as Base:

A solution of product from Example 32B (2 g, 6 mmol) in tetrahydrofuran (15 ml) was cooled to −20 to −22° C. under a nitrogen atmosphere. A solution of lithium hexamethyldisilazide (LiHMDS) (1.0M in THF, 7.2 ml, 7.2 mmol) was added, dropwise, maintaining the temperature at −20 to −22° C. After the addition, the solution was stirred at −20 to −22° C. for 2 hours. A solution of product from Example 7 (2.26 g, 8 mmol) in tetrahydrofuran (10 ml) was added, dropwise, at −20 to −22° C. The reaction was stirred for an additional 2.5 hours while maintaining the low temperature. The mixture was quenched with saturated ammonium chloride (15 ml) and extracted with ethyl acetate (3×10 ml). The organic extract was dried over anhydrous magnesium sulfate, filtered through silica pad and concentrated to a small residual volume. n-Heptane (10 ml) was add and the solution was left overnight at room temperature to produce 2.4 g (69%) of the product as white crystals. HPLC: 90% pure.

### EXAMPLE 34

### 4-[4-(4-Chlorophenoxy)benzenesulfonyl]-1-benzylpiperidine-4-carboxylic acid

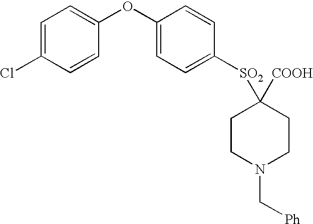

To a solution of product from Example 33 (30.7 g, HPLC 99.4 area %, 123 mmol) in t-butylmethylether (100 ml) at −25° C. was added a 2M lithium diisopropylamide solution (136 ml, 272 mmol) over a period of 15-20 minutes maintaining the temperature between −20 and −25° C. The yellow solution was stirred at this temperature for 2 hours. A solution of product from Example 7 (108 ml, 136 mmol) in tetrahydrofuran was added over a period of 15 minutes at −20° C. and the reaction was stirred for an additional hour while maintaining the low temperature. The reaction progress was monitored by thin layer chromatography, showing the formation of intermediate ester, Example 8. The reaction mixture was quenched with water and warmed to 20-25° C. while stirring for 0.5 hour. The organic solvent was removed by distillation (50 mm Hg, 35° C.) forming an oily layer on the bottom of the flask. Lithium hydroxide (15.5 g, 370 mmol) and methanol (150 ml) were added and the reaction mixture was heated at reflux temperature overnight (70° C.). The reaction mixture was clarified by filtration through filter paper to remove a small amount of gel-like insoluble material. The clarified solution was acidified with acetic acid (30 ml) at 20-25° C. to pH=5. Resulting slurry was stirred for 1 hour at ambient temperature and filtered. The solid residue was washed with water, slurried with ethanol (500 ml) for 0.5 hour, filtered and dried in vacuo at 40° C. to afford 36.4 g (61% HPLC) of the desired product as a yellow solid.

MS-ES: m/z 486 (M+H)⁺.

### EXAMPLE 35

### 4-[4-(4-Chlorophenoxy)benzenesulfonyl]-1-benzylpiperidine-4-carboxylic acid hydroxamide

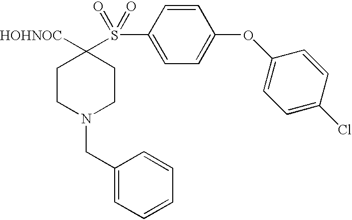

To a stirred suspension of the product from Example 34 (122.0 g, 0.251 mol) in acetonitrile (1.0 L) with a catalytic amount of dimethylformamide (1.0 ml) at 0° C. (ice bath) was added oxalyl chloride (55.1 g, 0.402 mol) over a period of 30 minutes (CAUTION: Gas evolution). The cooling bath was removed and the mixture was stirred at room temperature for 5 hour. (The reaction was monitored for completion by adding an aliquot of the reaction mixture to an excess of methanol followed by TLC, MS or HPLC). The acid chloride suspension was added, over a 20 minute period, to a cooled solution of powdered hydroxylamine hydrochloride (175.0 g, 2.51 mol) and triethylamine (330.9 g, 3.27 mol) in acetonitrile (2.5 L), which had been stirring for 3-5 hours at room temperature. The reaction temperature should not exceed ˜8° C. After stirring at room temperature for 18 hours, the reaction mixture was concentrated to afford an off-white residue. To the residue ethyl acetate (2.0 L) and water (2.0 L) were added, and the mixture was stirred for 15-20 minutes. The ethyl acetate layer was separated, filtered through anhydrous sodium sulfateand concentrated to give 130.4 g (crude yield 103%) as a semisolid product.

¹H NMR(DMSO-d₆, 300 MHz):δ10.9 (br s, 1H); 9.1 (br s, 1H); 7.71 (d, 2H, J=8.8 Hz); 7.52 (d, 2H, J=8.8 Hz); 7.33-7.19 (m, 7H); 7.14 (d, 2H, J=8.8 Hz); 3.4 (s, 2H); 2.7 (m, 2H); 2.28 (m, 2H); 1.95-1.8 (m, 4H);

HPLC: 94.06% product, (0.3% carboxylic acid and 2.88% mixed anhydride).

### EXAMPLE 36

### 4-[4-(4-Chlorophenoxy)benzenesulfonyl]-1-benzylpiperidine-4-carboxylic acid hydroxamide hydrochloride

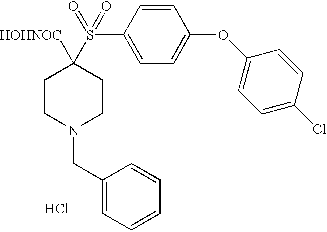

The crude product from Example 35 (130.4 g, 0.260 mol) was dissolved in ethyl acetate (350 ml) and conncentrated hydrochloric acid (31.3 ml, 0.313 mol) was added over a 20 minute period. Salts precipitated out of solution and the mixture was cooled in an ice bath at 2° C. for 30 minutes. The mixture was filtered, washed with cold (0° C.) ethyl acetate (50 ml×2), dried in an oven for 18 hours to give the product 118.6 g, (85%). This compound was recrystallized as follows:

A 5-L flask fitted with reflux condenser, thermometer/controller, and mechanical stirrer, was charged with ethanol (2.3 L, 200 proof) and the above crude product (118.6 g). The contents of the flask were heated at reflux temperature, then water (850 ml) was added over 60 minutes. The solution was clarified by filtration and reheated to boiling. The heating mantle was removed and the reaction mixture was cooled. Crystallization started at 60° C. The reaction was gradually cooled in an ice bath and kept at 2-4° C. for 30 minutes. The white crystals were collected, washed with cold ethanol (100 ml×2), dried in vacuo at 60° C. with a nitrogen bleed for 18 hours to give 89.23 g, (75%) of the desired product as crystals, m.p. 233-235° C.

HPLC: 98.5% pure; ¹H NMR(DMSO-d₆, 300 MHz):δ11.2 (s, 1H); 10.9 (br s, 1H); 9.35 (s, 1H); 7.73 (d, 2H, J=8.8 Hz); 7.52 (m, 4H0; 7.44 (br s, 3H); 7.23 (d, 2H, J=8.8 Hz); 7.17 (d, 2H, J=8.8 Hz); 4.26 (s, 2H0; 2.78 (m, 2H); 2.30 (m, 2H);

IR (KBr pellet): 3700-3300, 3156, 2931, 2543, 1677, 1483, 1244, 1144, 1087, 598 cm⁻¹.

### EXAMPLE 37

### 4-(4-but-2-ynyloxy-benzenesulfonyl)-piperidine-4-carboxylic acid methyl ester

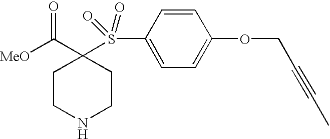

To a solution of product from Example 3 (500 mg, 1.11 mmol) in methylene chloride (10 ml) was added 4M HCl (2 ml) and the resulting mixture was stirred for 2 hours at room temperature. The solid was filtered, washed with ether to obtain 410 mg(95%) of the product as a solid.

IR: 3096, 2741, 2242, 1726, 1668, 1590, 1144, 836 cm⁻¹;

¹H NMR(300 MHz, CDCl₃):δ1.86(m, 3H), 2.52(m, 4H), 2.89(m, 2H), 3.52(m, 2H), 3.74(s, 3H), 4.74(m, 2H), 7.10(d, 2H, J=8.7 Hz), 7.69(d, 2H, J=8.7 Hz);

¹³C NMR(75 MHz, CDCl₃):δ3.6, 25.2, 41.2, 53.8, 56.9, 69.7, 72.6, 85.2, 115.4, 125.7, 132.3, 163.1, 166.6;

HR-MS:m/z Calculated for C₁₇H₂₁NO₅S 352.121; Found 352.1207.

### EXAMPLE 38

### 1-Acetyl-4-(4-but-2-ynyloxybenzenesulfonyl)piperidine-4-carboxylic acid methyl

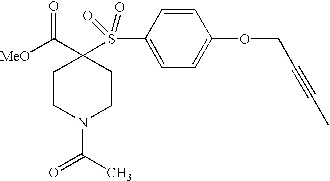

To a solution of product from Example 37 (105 mg, 0.23 mmol) in methylene chloride (1 ml) was added triethylamine (93 mg, 0.92 mmol), acetyl chloride(18 mg, 0.23 mmol) followed by a catalytic amount of dimethylaminopyridine. The resulting mixture was stirred for 8 hours at room temperature, quenched with water and extracted with methylene chloride. The organic layer was dried over anhydrous sodium sulfate and concentrated to give 75 mg (80%) of the product as a solid.

IR: 2928, 2242, 1726, 1636, 1595, 1451, 1302, 1150, 996 cm⁻¹;

¹H NMR(300 MHz, CDCl₃):δ1.87(t, 3H, J=2.4 Hz), 1.97-2.13(m, 2H), 2.09(s, 3H), 2.22-2.51(m, 3H), 3.02(m, 1H), 3.76(s, 3H), 3.89(m, 1H), 4.63(m, 1H), 4.74(q, 2H, J=2.4 Hz), 7.08(d, 2H, J=7.5 Hz), 7.14(d, 2H, J=7.5 Hz);

¹³C NMR(75 MHz, CDCl₃):δ4.1, 21.7, 28.4, 28.5, 38.9, 43.9, 53.7, 57.2, 72.7, 73.1, 85.5, 115.5, 126.9, 132.6, 163.1, 167.8, 169.2;

MS-ES: m/z 393.9 (M+H)⁺.

### EXAMPLE 39

### 1-Acetyl-4-(4-but-2-ynyloxybenzenesulfonyl)piperidine-4-carboxylic acid

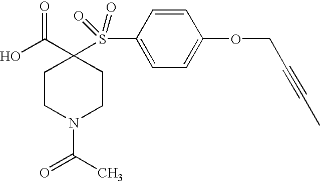

General procedure for step 3 was followed using product from Example 38 (240 mg, 0.61 mmol) in 4 ml of tetrahydrofuran: water (3:1), and lithium hydroxide (18 mg, 0.75 mmol) to obtain 200 mg(87%) of the acid.

IR: 2923, 2246, 1713, 1591, 1575, 1494, 1232, 994 cm⁻¹; ¹H NMR(300 MHz, acetone-d₆):δ1.84(t, 3H, J=2.8 Hz), 1.90-2.05(m, 2H), 2.06(s, 3H), 2.25-2.51(m, 3H), 3.06(m, 1H), 4.04(m, 1H), 4.63(m, 1H), 4.86(q, 1H, J=2.4 Hz), 7.18(d, 2H, J=8.4 Hz), 7.80(d, 2H, J=8.4 Hz);

¹³C NMR(75 MHz, CDCl₃):δ3.3, 21.3, 28.7, 39.0, 44.0, 57.4, 72.8, 74.2, 85.0, 115.8, 128.3, 133.4, 163.5, 168.4, 169.0;

HR-MS: m/z Calculated for C₁₈H₂₁NO₆S 380.1162; Found 380.1160.

### EXAMPLE 40

### 1-Acetyl-4-(4-but-2-ynyloxybenzenesulfonyl)piperidine-4-carboxylic acid hydroxamide

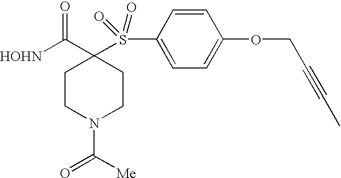

The general procedure for step 4 was followed using product from Example 39 (180 mg, 0.48 mmol) in dimethylformamide (4 ml), 1-hydroxybenzotriazole(77 mg, 0.57 mmol), 1-[3-(dimethylamino)propyl]-3-ethylcarbodiimide hydrochloride(127 mg, 0.66 mmol), N-methylmorpholine (0.078 ml, 0.71 mmol), and hydroxylamine (0.145 ml, 2.37 mmol) to obtain 100 mg(53%) of the product as a solid.

¹H NMR(300 MHz, CDCl₃):δ1.64(m, 1H), 1.85(m, 3H), 1.99(s, 3H), 2.31(m, 4H), 2.83(m, 1H), 3.88(m, 1H), 4.41(m, 1H), 4.88(m, 2H), 7.16(d, 2H, J=9.0 Hz), 7.66(d, 2H, J=9.0 Hz), 9.20(m, 1H), 11.00(m, 1H); ¹³C NMR(75 MHz, CDCl₃):δ3.5, 21.5, 36.1, 56.8, 70.2, 74.3, 84.7, 115.3, 126.7, 132.6, 162.3, 168.6;

MS-ES: m/z395.2 (M+H)⁺.

### EXAMPLE 41

### 1-Benzoyl-4-(4-but-2-ynyloxybenzenesulfonyl)piperidine-4-carboxylic acid methyl ester

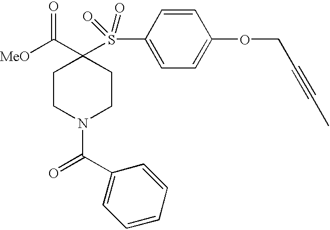

To a solution of product from Example 37 (400 mg, 1.03 mmol) in chloroform (10 ml) was added triethylamine (416 mg, 4.12 mmol), benzoyl chloride(144 μl, 1.24 mmol) followed by a catalytic amount of dimethylamino-pyridine. The resulting mixture was stirred for 15 hours at room temperature, quenched with water and extracted with methylene chloride. The organic layer was dried over anhydrous sodium sulfate and concentrated to give 375 mg (80%) of the product as a solid.

MS-ES: m/z 456.1 (M+H)⁺.

### EXAMPLE 42

### 1-Benzoyl-4-(4-but-2-ynyloxybenzenesulfonyl)piperidine-4-carboxylic acid

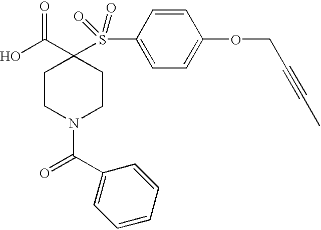

The general procedure for step 3 was followed using product from Example 41 (300 mg, 0.66 mmol) in 4 ml of tetrahydrofuran:water (3:1), and lithium hydroxide (18 mg, 0.75 mmol) to obtain 250 mg(86%) of the acid.

HR-MS: m/z Calculated for C₂₃H₂₃NO₆S 442.1319; Found 442.1317.

### EXAMPLE 43

### 1-Benzoyl-4-(4-but-2-ynyloxybenzenesulfonyl)piperidine-4-carboxylic acid hydroxamide

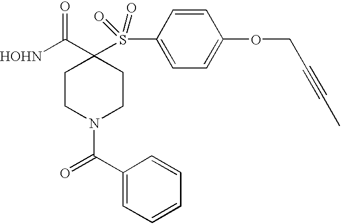

The general procedure for step 4 was followed using product from Example 42 (100 mg, 0.23 mmol) in dimethylformamide (2 ml), 1-hydroxybenzotriazole(36 mg, 0.27 mmol), 1-[3-(dimethylamino)propyl]-3-ethylcarbodiimide hydrochloride(62 mg, 0.32 mmol), N-methylmorpholine (0.038 ml, 0.35 mmol) and hydroxylamine (0.083 ml, 1.15 mmol) to obtain 40 mg(38%) of the product as a solid.

MS-ES: m/z 457.2 (M+H)⁺.

### EXAMPLE 44

### 1-(4-Methoxybenzoyl)-4-(4-but-2-ynyloxybenzenesulfonyl)piperidine-4-carboxylic acid methyl ester

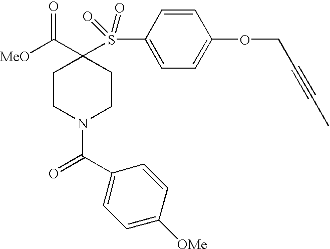

To a solution of product from Example 37 (260 mg, 0.77 mmol) in chloroform (7 ml) was added triethylamine (311 mg, 3.08 mmol), 4-methoxybenzoyl chloride(158 mg, 0.92 mmol) followed by a catalytic amount of dimethylaminopyridine. The resulting mixture was stirred for 15 hours at room temperature, quenched with water and extracted with methylene chloride. The organic layer was dried over anhydrous sodium sulfate and concentrated to give 280 mg (75%) of the product as a solid.

HR-MS: m/z Calculated for C₂₅H₂₇NO₇S 486.1581; Found 486.1576.

### EXAMPLE 45

### 1-(4-Methoxybenzoyl)-4-(4-but-2-ynyloxybenzenesulfonyl)piperidine-4-carboxylic acid

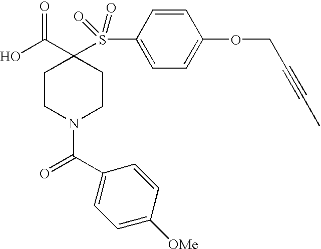

The general procedure for step 3 was followed using product from Example 44 (250 mg, 0.52 mmol) in 4 ml of tetrahydrofuran:methanol (1:1) and 1N sodium hydroxide (1.03 ml, 1.03 mmol) to obtain 150 mg(62%) of the acid.

HR-MS: m/z Calculated for C₂₄H₂₅NO₇S 472.1425; Found 472.1426.

### EXAMPLE 46

### 1-(4-Methoxybenzoyl)-4-(4-but-2-ynyloxybenzenesulfonyl)piperidine-4-carboxylic acid hydroxamide

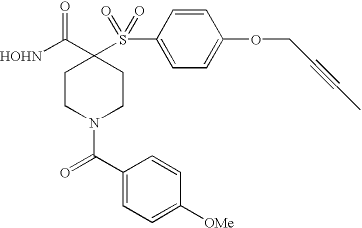

The general procedure for step 4 was followed using product from Example 45 (90 mg, 0.19 mmol) in dimethylformamide (2 ml), 1-hydroxybenzotriazole(31 mg, 0.23 mmol), 1-[3-(dimethylamino)propyl]-3-ethylcarbodiimide hydrochloride(51 mg, 0.27 mmol), N-methylmorpholine (0.031 ml, 0.28 mmol) and hydroxylamine (0.068 ml, 0.95 mmol) to obtain 70 mg(76%) of the product as a solid.

HR-MS: m/z Calculated for C₂₄H₂₆N₂O₇S 487.1534; Found 487.1531.

### EXAMPLE 47

### 4-(4-But-2-ynyloxybenzenesulfonyl)-1-(pyrrolidine-1-carbonyl)piperidine-4-carboxylic acid methyl ester

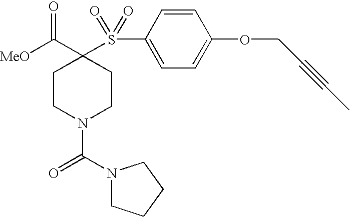

To a solution of product from Example 37 (400 mg, 1.03 mmol) in chloroform (10 ml) was added triethylamine (208 mg, 2.06 mmol), pyrrolidine-carbonyl chloride (206 mg, 1.54 mmol) followed by a catalytic amount of dimethylaminopyridine. The resulting mixture was stirred for 15 hours at room temperature, quenched with water and extracted with methylene chloride. The organic layer was dried over anhydrous sodium sulfate and concentrated to give 400 mg (87%) of the product as a solid.

MS-ES: m/z 449.3 (M+H)⁺.

### EXAMPLE 48

### 4-(4-but-2-ynyloxybenzenesulfonyl)-1-(pyrrolidine-1-carbonyl)-piperidine-4-carboxylic acid

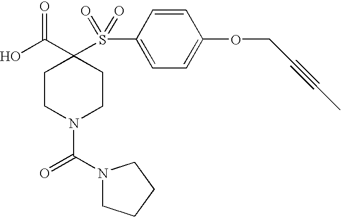

The general procedure for step 3 was followed using product from Example 47 (400 mg, 0.89 mmol) in 4 ml of tetrahydrofuran: methanol; water (1:1:0.5) and lithium hydroxide (48 mg, 2.0 mmol) to obtain 300 mg(78%) of the acid.

MS-ES: m/z 435.2 (M+H)⁺.

### EXAMPLE 49

### 4-(4-But-2-ynyloxybenzenesulfonyl)-N-hydroxy-1-(pyrrolidine-1-carbonyl)-4-piperidinecarboxamide

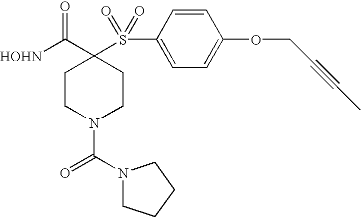

The general procedure for step 4 was followed using product from Example 48 (255 mg, 0.23 mmol) in dimethylformamide (6 ml), 1-hydroxybenzotriazole(96 mg, 0.71 mmol), 1-[3-(dimethylamino)propyl]-3-ethylcarbodiimide hydrochloride (157 mg, 0.82 mmol), N-methylmorpholine (0.099 ml, 0.84 mmol) and hydroxylamide (0.181 ml, 2.8 mmol) to obtain 150 mg(60%) of the product as a solid.

HR-MS: m/z Calculated for C₂₁H₂₇N₃O₆S 450.1693; Found 450.1692.

### EXAMPLE 50

### 1-Ethyl 4-methyl 4-(4-but-2-ynyloxybenzenesulfonyl)-1,4-piperidinedicarboxylate

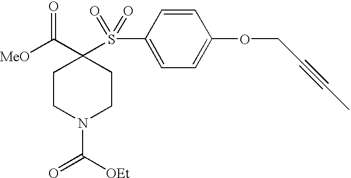

To a solution of product from Example 37 (400 mg, 1.03 mmol) in chloroform (10 ml) was added sodium bicarbonate (865 mg, 10.3 mmol) and ethylchloroformate (0.147 ml, 1.54 mmol). The resulting mixture was stirred for 15 hours at room temperature, quenched with water and extracted with methylene chloride. The organic layer was dried over anhydrous sodium sulfate and concentrated to give 425 mg (98%) of the product as a solid.

MS-ES: m/z 424.4 (M+H)⁺.

### EXAMPLE 51

### 1-(Ethylcarbonyl)-4-(4-but-2-ynyloxybenzenesulfonyl)-1-piperidinecarboxylic acid

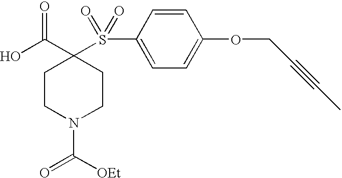

The general procedure for step 3 was followed using product from Example 50 (400 mg, 0.95 mmol) in 8 ml of tetrahydrofuran: methanol: water (1:1:0.5) and lithium hydroxide (50 mg, 2.04 mmol) to obtain 340 mg(88%) of the acid.

HR-MS: m/z Calculated for C₁₉H₂₃NO₇S 408.1122; Found 408.1126.

### EXAMPLE 52

### Ethyl 4-(4-but-2-ynyloxybenzenesulfonyl)-4-[(hydroxyamino)carbonyl]-1-piperidinecarboxylate

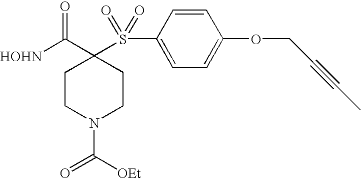

The general procedure for step 4 was followed using product from Example 51 (225 mg, 0.55 mmol) in dimethylformamide (5 ml), 1-hydroxybenzotriazole(89 mg, 0.66 mmol), 1-[3-(dimethylamino)propyl]-3-ethylcarbodiimide hydrochloride (148 mg, 0.77 mmol), N-methylmorpholine (0.091 ml, 0.86 mmol) and hydroxylamine (0.168 ml, 2.75 mmol) to obtain 150 mg(64%) of the product as a solid.

HR-MS: m/z Calculated for C₁₉H₂₄N₂O₇S 425.1377; Found 425.1375.

### EXAMPLE 53

### Methyl 4-(4-but-2-ynyloxybenzenesulfonyl)-1-[(trifluoromethyl)sulfonyl]-4-piperidinecarboxylate

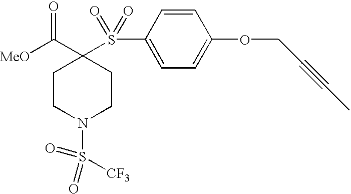

To a solution of product from Example 37 (350 mg, 0.90 mmol) in chloroform (10 ml) was added triethylamine (182 mg, 1.81 mmol) and trifluoromethanesulfonyl chloride(0.125 ml, 1.17 mmol) followed by a catalytic amount of dimethylaminopyridine. The resulting mixture was stirred for 15 hours at room temperature, quenched with water and extracted with methylene chloride. The organic layer was dried over anhydrous sodium sulfate and concentrated to give 245 mg (56%) of the product as a solid.

HR-MS: m/z Calculated for C₁₈H₂₀F₃NO₇S₂ 484.0706; Found 484.0700.

### EXAMPLE 54

### 4-(4-But-2-ynyloxybenzenesulfonyl)-1-[(trifluoromethyl)sulfonyl]-4-piperidinecarboxylic acid

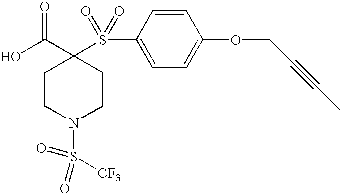

The general procedure for step 3 was followed using product from Example 53 (225 mg, 0.47 mmol) in 5 ml of tetrahydrofuran: methanol; water (1:1:0.5) and lithium hydroxide (24 mg, 0.98 mmol) to obtain 175 mg(80%) of the acid.

MS-ES: m/z 468.1 (M−H)⁻.

### EXAMPLE 55

### 4-(4-But-2-ynyloxybenzenesulfonyl)-N-hydroxy-1-[(trifluoromethyl)sulfonyl]-4-piperidinecarboxamide

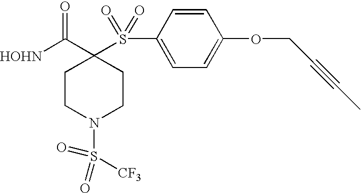

The general procedure for step 4 was followed using product from Example 54 (145 mg, 0.31 mmol) in dimethylformamide (3 ml), 1-hydroxybenzotriazole(50 mg, 0.37 mmol), 1-[3-(dimethylamino)propyl]-3-ethylcarbodiimide hydrochloride(83 mg, 0.47 mmol), N-methylmorpholine (0.051 ml, 0.47 mmol) and hydroxylamine (0.095 ml, 1.55 mmol) to obtain 90 mg(60%) of the product as a solid.

HR-MS: m/z Calculated for C₁₇H₁₉F₃N₂O₇S₂ 485.0659; Found 485.0666.

### EXAMPLE 56

### Methyl 4-(4-but-2-ynyloxybenzenesulfonyl)-1-(3-pyridinylcarbonyl)-4-piperidinecarboxylate

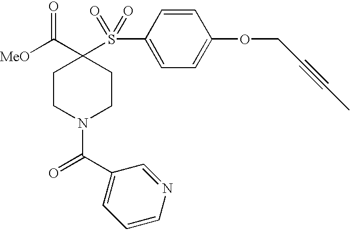

To a solution of product from Example 37 (500 mg, 1.29 mmol) in methylene chloride (10 ml) was added triethylamine (443 mg, 4.39 mmol) and nicotinyl chloride (276 ml, 1.55 mmol) followed by a catalytic amount of dimethylaminopyridine. The resulting mixture was stirred for 15 hours at room temperature, quenched with water and extracted with methylene chloride. The organic layer was dried over anhydrous sodium sulfate and concentrated to give 460 mg (78%) of the product as a solid.

HR-MS: m/z Calculated for C₂₃H₂₄N₂O₆S 457.1428; Found 457.1428.

### EXAMPLE 57

### 4-(4-But-2-ynyloxybenzenesulfonyl)-1-(3-pyridinylcarbonyl)-4-piperidinecarboxylic acid

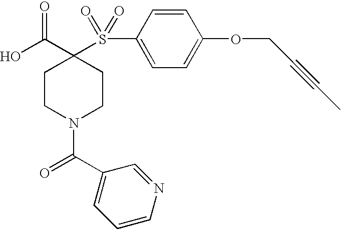

The general procedure for step 3 was followed using product from Example 56 (430 mg, 0.94 mmol) in 8 ml of tetrahydrofuran: methanol (1:1) and 1N sodium hydroxide (1.89 ml, 1.89 mmol) to obtain 235 mg(57%) of the acid.

HR-MS: m/z Calculated for C₂₂H₂₂N₂O₆S 443.1271; Found 443.1270.

### EXAMPLE 58

### 4-(4-But-2-ynyloxybenzenesulfonyl)-N-hydroxy-1-(3-pyridinylcarbonyl)-4-piperidinecarboxamide

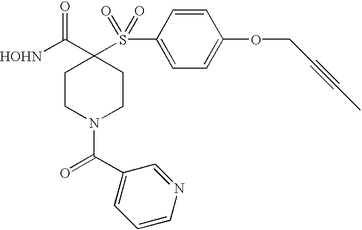

The general procedure for step 4 was followed using product from Example 57 (195 mg, 0.44 mmol) in dimethylformamide (4 ml), 1-hydroxybenzotriazole(72 mg, 0.53 mmol), 1-[3-(dimethylamino)propyl]-3-ethylcarbodiimide hydrochloride (119 mg, 0.62 mmol), N-methylmorpholine (0.072 ml, 0.66 mmol) and hydroxylamine (0.135 ml, 2.2 mmol) to obtain 65 mg(32%) of the product as a solid.

HR-MS: m/z Calculated for C₂₂H₂₃N₃O₆S 458.1380; Found 458.1373.

### EXAMPLE 59

### Methyl 4-(4-but-2-ynyloxybenzenesulfonyl)-1-(2-thienylcarbonyl)-4-piperidinecarboxylate

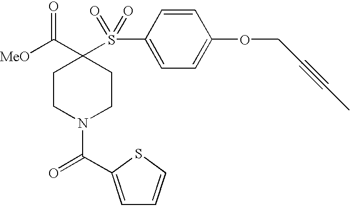

To a solution of product from Example 37 (500 mg, 1.29 mmol) in methylene chloride (10 ml) was added triethylamine (261 mg, 2.58 mmol) and thiophenylcarbonyl chloride (227 mg, 1.55 mmol) followed by a catalytic amount of dimethylaminopyridine. The resulting mixture was stirred for 15 hours at room temperature, quenched with water and extracted with methylene chloride. The organic layer was dried over anhydrous sodium sulfate and concentrated to give 480 mg (81%) of the product as a solid.

HR-MS: m/z Calculated for C₂₂H₂₃NO₆S₂ 462.1040; Found 462.1039.

### EXAMPLE 60

### 4-(4-But-2-ynyloxybenzenesulfonyl)-1-(2-thienylcarbonyl)-4-piperidinecarboxylic acid

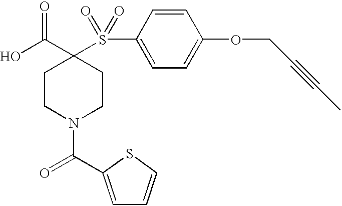

The general procedure for step 3 was followed using product from Example 59 (435 mg, 0.94 mmol) in 8 ml of tetrahydrofuran: methanol (1:1) and 1N sodium hydroxide (1.89 ml, 1.89 mmol) to obtain 360 mg(86%) of the acid.

HR-MS: m/z Calculated for C₂₁H₂₁NO₆S₂ 448.0883; Found 448.0882.

### EXAMPLE 61

### 4-(4-but-2-ynyloxybenzenesulfonyl)-N-hydroxy-1-(2-thienylcarbonyl)-4-piperidinecarboxamide

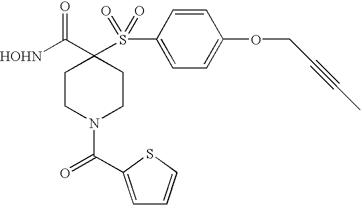

The general procedure for step 4 was followed using product from Example 60 (335 mg, 0.75 mmol) in dimethylformamide (7 ml), 1-hydroxybenzotriazole(121 mg, 0.90 mmol), 1-[3-(dimethylamino)propyl]-3-ethylcarbodiimide hydrochloride (201 mg, 1.05 mmol), N-methylmorpholine (0.124 ml, 1.13 mmol) and hydroxylamine (0.229 ml, 3.75 mmol) to obtain 216 mg(62%) of the product as a solid.

HR-MS: m/z Calculated for C₂₁H₂₂N₂O₆S₂ 463.0992; Found 463.0988.

### EXAMPLE 62

### Methyl 4-(4-but-2-ynyloxybenzenesulfonyl)-1-[(4-methoxyphenyl)sulfonyl]-4-piperidinecarboxylate

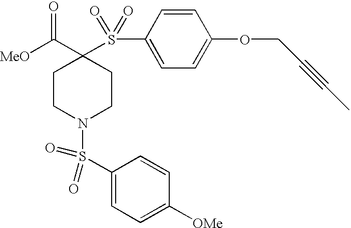

To a solution of product from Example 37 (500 mg, 1.29 mmol) in methylene chloride(10 ml) was added triethylamine (261 mg, 2.58 mmol) and 4-methoxyphenylsulfonyl chloride(320 mg, 1.55 mmol) followed by a catalytic amount of dimethylaminopyridine. The resulting mixture was stirred for 15 hours at room temperature, quenched with water and extracted with methylene chloride. The organic layer was dried over anhydrous sodium sulfate and concentrated to give 590 mg (88%) of the product as a solid.

HR-MS: m/z Calculated for C₂₄H₇₂NO₈S₂ 522.1251; Found 522.1252.

### EXAMPLE 63

### 4-(4-but-2-ynyloxybenzenesulfonyl)-1-[(4-methoxyphenyl)sulfonyl]-4-piperidinecarboxylic acid

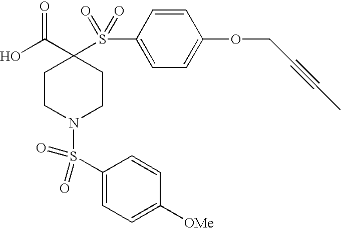

The general procedure for step 3 was followed using product from Example 62 (545 mg, 1.04 mmol) in 8 ml of tetrahydrofuran: methanol (1:1) and 1N sodium hydroxide (2.09 ml, 2.09 mmol) to obtain 446 mg(85%) of the acid.

HR-MS: m/z Calculated for C₂₃H₂₅NO₈S₂ 508.1094; Found 508.1073.

### EXAMPLE 64

### 4-(4-but-2-ynyloxybenzenesulfonyl)-N-hydroxy-1-[(4-methoxyphenyl)sulfonyl]-4-piperidinecarboxamide

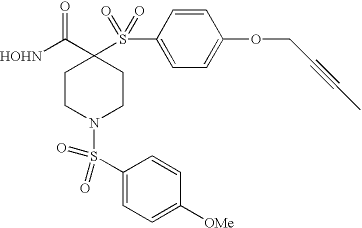

The general procedure for step 4 was followed using product from Example 63 (402 mg, 0.79 mmol) in dimethylformamide (8 ml), 1-hydroxybenzotriazole(128 mg, 0.95 mmol), 1-[3-(dimethylamino)propyl]-3-ethylcarbodiimide hydrochloride (212 mg, 1.11 mmol), N-methylmorpholine (0.130 ml, 1.19 mmol) and hydroxylamine (0.242 ml, 3.95 mmol) to obtain 396 mg(96%) of the product as a solid.

HR-MS: m/z Calculated for C₂₃H₂₆N₂O₈S₂ 523.1203; Found 523.1198.

### EXAMPLE 65

### Methyl 4-(4-but-2-ynyloxybenzenesulfonyl)-1-[(2,2,5-trimethyl-1,3-dioxan-5-yl)carbonyl]-4-piperidinecarboxylate

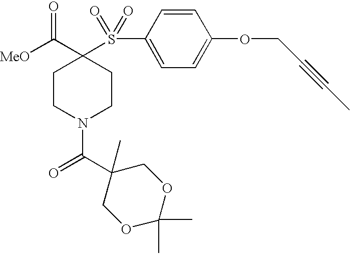

The general procedure for step 4 was followed using product from Example 37 (500 mg, 1.29 mmol) in dimethylformamide (10 ml), (2,2,5-trimethyl-1,3-dioxan-5-yl)carboxylic acid (224 mg, 1.29 mmol), 1-hydroxybenzotriazole(209 mg, 1.56 mmol), 1-[3-(dimethylamino)propyl]-3-ethylcarbodiimide hydrochloride(346 mg, 1.81 mmol) and N-methylmorpholine (0.212 ml, 1.94 mmol) to obtain 385 mg(59%) of the product as a solid.

HR-MS: m/z Calculated for C₂₅H₃₃NO₈S 508.2000; Found 508.1998.

### EXAMPLE 66

### 4-(4-But-2-ynyloxybenzenesulfonyl)-1-[(2,2,5-trimethyl-1,3-dioxan-5-yl)carbonyl]-4-piperidinecarboxylic acid

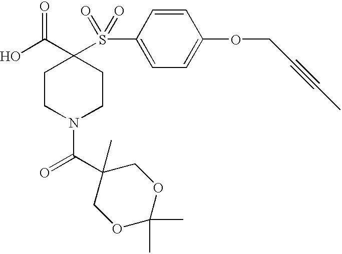

The general procedure for step 3 was followed using product from Example 65 (335 mg, 0.66 mmol) in 4 ml of tetrahydrofuran: methanol (1:1) and 1N sodium hydroxide (1.3 ml, 1.3 mmol) to obtain 315 mg(97%) of the acid.

HR-MS: m/z Calculated for C₂₄H₃₁NO₈S 494.1843; Found 494.1835.

### EXAMPLE 67

### 4-(4-but-2-ynyloxybenzenesulfonyl)-N-hydroxy-1-[2,2,5-trimethyl-1,3-dioxan-5-yl)carbonyl]-4-piperidinecarboxamide

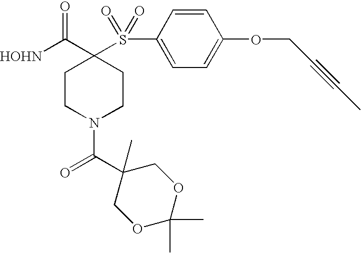

The general procedure for step 4 was followed using product from Example 66 (280 mg, 0.57 mmol) in dimethylformamide (6 ml), 1-hydroxybenzotriazole(92 mg, 0.68 mmol), 1-[3-(dimethylamino)propyl]-3-ethylcarbodiimide hydrochloride(153 mg, 0.80 mmol), N-methylmorpholine (0.094 ml, 0.85 mmol) and hydroxylamine (0.174 ml, 2.85 mmol) to obtain 180 mg( 62%) of the product as a solid.

HR-MS: m/z Calculated for C₂₄H₃₂N₂O₈S 531.1771; Found 531.1768.

### EXAMPLE 68

### 4-(4-but-2-ynyloxybenzenesulfonyl)-N-hydroxy-1-[3-hydroxy-2-(hydroxymethyl)-2-methylpropanoyl]-4-piperidinecarboxamide

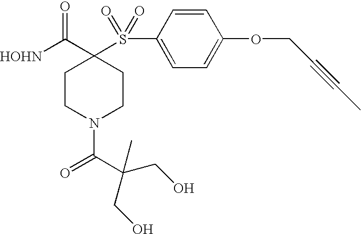

To a solution of product from Example 67 (150 mg, 0.29 mmol) in tetrahydrofuran (2 ml) was added 1N aqueous hydrochloric acid (2 ml) and the resulting mixture was stirred for 4 hours. The organic layer was washed with sodium bicarbonate, saturated sodium chloride solution and dried over anhydrous sodium sulfate. The organic solvent was concentrated to obtain 40 mg (29%) of the product.

HR-MS: m/z Calculated for C₂₁H₂₈N₂O₈S 469.1639; Found 469.1637.

### EXAMPLE 69

### Methyl ({4-{[4-(2-butynyloxy)phenyl]sulfonyl}-4-[(hydroxyamino)carbonyl]-1-piperidinyl}methyl)benzoate hydrochloride

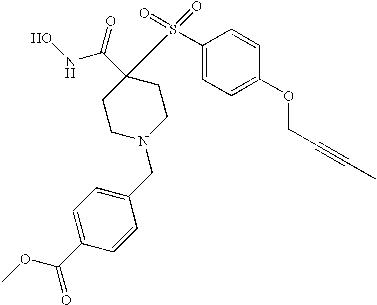

To a solution of product from Example 6 (2.5 g, 6.43 mmol) and methyl 4-(bromomethyl)benzoate (1.62 g, 7.07 mmol) in methanol (100 ml) at 50° C. was added triethylamine (2.25 ml, 16.1 mmol). After 30 minutes additional methanol (50 ml) was added. The reaction mixture was stirred for 18 hours, concentrated in vacuo and 1N aqueous hydrochloric acid (10 ml) and water were added. The resulting solid was isolated and methanol (20 ml) and 1N hydrochloric acid in diethyl ether (15 ml) were added. Additional diethyl ether was added followed by trituration of the precipitate to give the desired product as a white powder (2.4 g).

¹H NMR (DMSO-d₆, 300 MHz):δ1.85 (t, 3H, CH3, J=2.2 Hz), 2.1-3.5 (m, 8H), 3.87 (S, 3H), 4.40 (bd s, 2H), 4.89 (q, 2H, J=2.2 Hz), 7.1-8.1 (m, 8H), 9.3-11.2(m, 3H); MS-ES: m/z 501.5 (M+H)⁺.

The subject compounds of the present invention may be tested for biological activity according to the following procedures.

### In Vitro Gelatinase Assay

The assay is based on the cleavage of the thiopeptide substrate ((Ac-Pro-Leu-Gly(2 mercapto-4 methyl-pentanoyl)-Leu-Gly-OEt), Bachem Bioscience) by the enzyme, gelatinase, releasing the substrate product which reacts calorimetrically with DTNB ((5,5′-dithio-bis(2-nitro-benzoic acid)). The enzyme activity is measured by the rate of the color increase.

The thiopeptide substrate is made up fresh as a 20 mM stock in 100% DMSO and the DTNB is dissolved in 100% DMSO as a 100 mM stock and stored in dark at room temperature. Both the substrate and DTNB are diluted together to 1 mM with substrate buffer (50 mM HEPES pH 7.5, 5 mM CaCl₂) before use. The stock of human neutrophil gelatinase B is diluted with assay buffer (50 mM HEPES pH 7.5, 5 mM CaCl₂, 0.02% Brij) to a final concentration of 0.15 nM. The assay buffer, enzyme, DTNB/substrate (500 μM final concentration) and vehicle or inhibitor are added to a 96 well plate (total reaction volume of 200 μl ) and the increase in color is monitored spectrophotometrically for 5 minutes at 405 nm on a plate reader.

The increase in OD₄₀₅ is plotted and the slope of the line is calculated which represents the reaction rate.

The linearity of the reaction rate is confirmed (r²>0.85). The mean (x±sem) of the control rate is calculated and compared for statistical significance (p<0.05) with drug-treated rates using Dunnett's multiple comparison test. Dose-response relationships can be generated using multiple doses of drug and IC₅₀ values with 95% CI are estimated using linear regression (IPRED, HTB).

References: Weingarten, H and Feder, J., Spectrophotometric assay for vertebrate collagenase, Anal. Biochem. 147, 437-440 (1985).

### In Vitro Collagenase Assay

The assay is based on the cleavage of a peptide substrate ((Dnp-Pro-Cha-Gly-Cyns(Me)-His-Ala-Lys(NMa)-NH₂), Peptide International, Inc.) by collagenase releasing the fluorescent NMa group which is quantitated on the fluorometer. Dnp quenches the NMa fluorescence in the intact substrate. The assay is run in HCBC assay buffer (50 mM HEPES, pH 7.0, 5 mM Ca⁺², 0.02% Brij, 0.5% Cysteine), with human recombinant fibroblast collagenase (truncated, mw=18,828, WAR, Radnor). Substrate is dissolved in methanol and stored frozen in 1 mM aliquots. Collagenase is stored frozen in buffer in 25 μM aliquots. For the assay, substrate is dissolved in HCBC buffer to a final concentration of 10 μM and collagenase to a final concentration of 5 nM. Compounds are dissolved in methanol, DMSO, or HCBC. The methanol and DMSO are diluted in HCBC to <1.0%. Compounds are added to the 96 well plate containing enzyme and the reaction is started by the addition of substrate.

The reaction is read (excitation 340 nm, emission 444 nm) for 10 min. and the increase in fluorescence over time is plotted as a linear line. The slope of the line is calculated and represents the reaction rate.

The linearity of the reaction rate is confirmed (r²>0.85). The mean (x±sem) of the control rate is calculated and compared for statistical significance (p<0.05) with drug-treated rates using Dunnett's multiple comparison test. Dose-response relationships can be generated using multiple doses of drug and IC₅₀ values with 95% CI are estimated using linear regression (IPRED, HTB).

References: Bickett, D. M. et al., A high throughput fluorogenic substrate for interstitial collagenase (MMP-1) and gelatinase (MMP-9), Anal. Biochem. 212,58-64 (1993).

### Procedure for Measuring TACE Inhibition

Using 96-well black microtiter plates, each well receives a solution composed of 10 μL TACE (Immunex, final concentration 1 μg/mL), 70 μL Tris buffer, pH 7.4 containing 10% glycerol (final concentration 10 mM), and 10 μL of test compound solution in DMSO (final concentration 1 μM, DMSO concentration <1%) and incubated for 10 minutes at room temperature. The reaction is initiated by addition of a fluorescent peptidyl substrate (final concentration 100 μM) to each well and then shaking on a shaker for 5 sec.

The reaction is read (excitation 340 nm, emission 420 nm) for 10 min. and the increase in fluorescence over time is plotted as a linear line. The slope of the line is calculated and represents the reaction rate.

The linearity of the reaction rate is confirmed (r²>0.85). The mean (x±sem) of the control rate is calculated and compared for statistical significance (p<0.05) with drug-treated rates using Dunnett's multiple comparison test. Dose-response relationships can be generate using multiple doses of drug and IC₅₀ values with 95% CI are estimated using linear regression.

The compound of Example 18 was found to inhibit MMPs and TACE as follows:

TACE inhibition (at 10 uM): 54%;

MMP1 (1C50): 1.3 uM;

MMP9 (IC50): 0.732 uM;

MMP13 (IC50): 0.14 uM.

Thus compounds of the present invention are useful inhibitors of MMPs and TACE.

### Pharmaceutical Composition

Compounds of this invention may be administered neat or with a pharmaceutical carrier to a patient in need thereof. The pharmaceutical carrier may be solid or liquid.

Applicable solid carriers can include one or more substances which may also act as flavoring agents, lubricants, solubilizers, suspending agents, fillers, glidants, compression aids, binders or tablet-disintegrating agents or an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such a solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g., cellulose derivatives, preferable sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration.

Liquid pharmaceutical compositions which are sterile solutions or suspensions can be utilized by, for example, intramuscular, intraperitoneal or subcutaneous injection. Sterile solutions can also be administered intravenously. Oral administration may be either liquid or solid composition form.

The compounds of this invention may be administered rectally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may also be administered transdermally through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semi-solid emulsions of either the oil in water or water in oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

The dosage to be used in the treatment of a specific patient suffering from a disease or condition in which MMPs and TACE are involved must be subjectively determined by the attending physician. The variables involved include the severity of the dysfunction, and the size, age, and response pattern of the patient. Treatment will generally be initiated with small dosages less than the optimum dose of the compound. Thereafter the dosage is increased until the optimum effect under the circumstances is reached. Precise dosages for oral, parenteral, nasal, or intrabronchial administration will be determined by the administering physician based on experience with the individual subject treated and standard medical principles.

Preferably the pharmaceutical composition is in unit dosage form, e.g., as tablets or capsules. In such form, the composition is sub-divided in unit dose containing appropriate quantities of the active ingredient; the unit dosage form can be packaged compositions, for example packed powders, vials, ampoules, prefilled syringes or sachets containing liquids. The unit dosage form can be, for example, a capsule or tablet itself, or it can be the appropriate number of any such compositions in package form.

1. A method of preparing alpha-sulfonyl derivatives of the formula V: 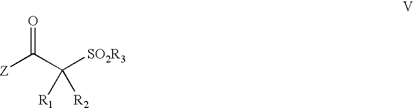
wherein
Z is H, OH, —NYOX, —OR₅ or —NR₅R₆;
X is hydrogen, alkyl of 1-6 carbon atoms, benzyl, hydroxyethyl, t-butyldimethylsilyl, trimethylsilyl or tetrahydropyranyl;
Y is hydrogen, alkyl of 1-6 carbon atoms, aryl of 6 to 10 carbon atoms, 5-10 membered heteroaryl having 1-3 heteroatoms selected from N, NR₄, O and S, cycloalkyl of 3-6 carbon atoms, 5-10 membered cycloheteroalkyl; wherein said alkyl, aryl, heteroaryl, cycloalkyl and cycloheteroalkyl group of Y is optionally substituted on any atom capable of substitution, with 1 to 3 substituents selected from the group consisting of halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅, perfluoroalkyl of 1-4 carbon atoms, —O-perfluoroalkyl of 1-4 carbon atoms, —CONR₅R₆,—S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅ COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;
R₁ and R₂ are each, independently, hydrogen; aryl of 6 to 10 carbon atoms; 5-10 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms having 1 to 3 double bonds; alkynyl of 2-18 carbon atoms having from 1 to 3 triple bonds; or R₁ and R₂ taken together with the carbon atom to which they are attached form a cycloalkyl ring of 3-8 carbon atoms or a 5-10 membered cycloheteroalkyl ring; and wherein the aryl, heteroaryl, cycloalkyl, cycloheteroalkyl, alkyl, alkenyl, and alkynyl, may be optionally substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅, perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆,—NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;
R₃ is alkyl of 1-18 carbon atoms, alkenyl of 2-18 carbon atoms having 1 to 3 double bonds, alkynyl of 2-18 carbon atoms having from 1 to 3 triple bonds, cycloalkyl of 3-6 carbon atoms, 5-10 membered cycloheteroalkyl, aryl of 6 to 10 carbon atoms, 5-6 membered heteroaryl having 1-3 heteroatoms selected from N, NR₄, O, and S; wherein said alkyl, alkenyl, alkynyl, cycloalkyl, cycloheteroalkyl, aryl and heteroaryl of R₃ may optionally be substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅, perfluoroalkyl of 1-4 carbon atoms, —O-perfluoroalkyl of 1-4 carbon atoms, —CONR₅R₆, —S(O)ₙR₅, —-OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;
R₄ is hydrogen; aryl; aralkyl, heteroaryl; heteroaralkyl, alkyl of 1-6 carbon atoms; cycloalkyl of 3-6 carbon atoms; —C(O)ₙR₅, —CONR₅R₆ or SO₂R₅; p1 R₅ and R₆ are each independently hydrogen, optionally substituted aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms or alkynyl of 2-18 carbon atoms; or R₅ and R₆ taken together with the nitrogen atom to which they are attached may form a 5-10 membered cycloheteroalkyl ring; and
n is 1 or 2; or a pharmaceutical salt thereof,
which comprises reacting a sulfonyl fluoride of the formula III
R₃′SO₂F III
wherein R₃′ is as hereinabove defined for R₃ with the proviso that R₃′ does not contain a group that can form an anion under basic conditions; with a carbonyl compound of the formula IV: 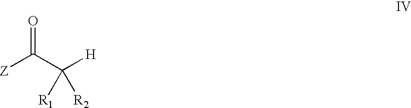
wherein Z is H, OH, YNOX, —NR₅R₆ or OR₅, and X, Y, R₁, R₂, R₅, and R₆ are as hereinabove defined; in the presence of a metal hydride or amide base in an ether organic solvent at temperatures from about −78° C. to about 30° C. to produce an alpha-sulfonyl carbonyl compound of formula V;
any reactive substituent group(s) being protected during the reaction and removed thereafter; and further if desired isolating any chiral or stereoisomeric product as an individual isomer.

2. A method as claimed in claim 1 in which the compound of formula (V) prepared wherein Z is H, OH, —NR₅R₆ or OR₅ is further reacted to convert it to an alpha-sulfonyl hydroxamic acid derivative of the formula I: 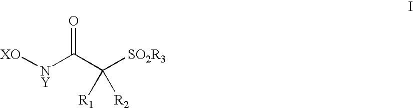
wherein X, Y, R₁, R₂ and R₃ are as defined in claim 1 or a pharmaceutically acceptable salt thereof; any reactive substituent group(s) being protected during the reaction and removed thereafter; and further if desired isolating any chiral or stereoisomeric product as an individual isomer.

3. A method as claimed in claim 2 wherein Z in the compound of formula V prepared is:
(i) OR₅ wherein R₅ is other than hydrogen and the conversion to the alpha-sulfonyl hydroxamic acid derivative of the formula I is carried out by:
a) reacting the compound of formula V with an alkali metal hydroxide in the presence of water, and/or ether organic solvent or alcohol at temperatures ranging from about 0° C. to about 100° C. to produce a carboxylic acid of the formula VI: 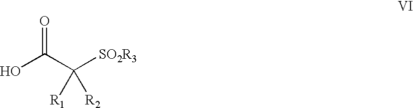
wherein, R₁, R₂, and R₃ are as hereinabove defined; and
(b) reacting the carboxylic acid of formula VI with a hydroxylamine or hydroxylamine derivative of the formula VII:
XONHY VII
wherein X and Y are as hereinabove defined; in the presence of suitable coupling reagent and polar organic solvent to produce a hydroxamate of the formula I or
(ii) OH and the conversion to the alpha-sulfonyl hydroxamic acid derivative of the formula I is carried out according to step b) above.

4. The method of claim 3 wherein the ether organic solvent in step a) is selected from tetrahydrofuran, diethylether and dioxane.

5. The method of claim 3 wherein the alcohol in step a) is selected from methanol and ethanol.

6. The method of claim 3 wherein the alkali metal hydroxide in step a) is selected from lithium hydroxide and sodium hydroxide.

7. The method of claim 3 wherein the polar organic solvent in step b) is dimethylformamide.

8. The method of claim 3 wherein the coupling reagent is selected from the group consisting of 1-(3-dimethylaminopropyl)-3-ethylcarbodimide hydrochloride, N-hydroxybenzotriazole, N-methylmorpholine and oxalylchloride and triethylamine.

9. The method of claim 3 wherein the coupling reaction is carried out at a temperature from about 0° C. to 30° C.

10. The method of claim 3 wherein the ether organic solvent used in the reaction between the compounds of formula III and IV is selected from tetrahydrofuran, diethylether and dioxane.

11. The method of claim 3 wherein the metal hydride base or amide base used in the reaction between the compounds of formula III and IV and is selected from lithium diisopropylamine, lithiumhexamethyldisilazide, and sodium hydride.

12. The method of claim 1 wherein the sulfonyl fluoride of formula III is prepared by reacting a sulfonyl chloride of the formula II
R₃′SO₂Cl II
wherein R₃′ is as defined for R₃ in claim 1 with the proviso that R₃′ does not contain a group that can form an anion under basic conditions, with a fluorinating agent in the presence of a polar organic solvent from about 15° C. to about 30° C.

13. The method of claim 12 wherein the fluorinating agent is selected from potassium fluoride, potassium fluoride-calcium fluoride mixture and cesium fluoride.

14. The method of claim 12 wherein the polar organic solvent is selected from acetonitrile and tetrahydrofuran.

15. A method of preparing alpha-sulfonyl derivatives of the formula V: 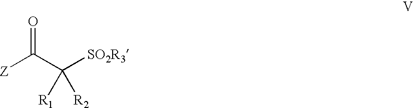
wherein
Z is H, OH, —NYOX, —OR₅ or —NR₅R₆;
R₁ and R₂ are each, independently, hydrogen; aryl of 6 to 10 carbon atoms; 5-10 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms having 1 to 3 double bonds; alkynyl of 2-18 carbon atoms having from 1 to 3 triple bonds; or R₁ and R₂ taken together with the carbon atom to which they are attached form a cycloalkyl ring of 3-8 carbon atoms or a 5-10 membered cycloheteroalkyl ring; and wherein the aryl, heteroaryl, cycloalkyl, cycloheteroalkyl, alkyl, alkenyl, and alkynyl, may be optionally substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅, perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, 13 NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;
R₃′ is alkyl of 1-18 carbon atoms, alkenyl of 2-18 carbon atoms having 1 to 3 double bonds, alkynyl of 2-18 carbon atoms having from 1 to 3 triple bonds, cycloalkyl of 3-6 carbon atoms, 5-10 membered cycloheteroalkyl, aryl of 6 to 10 carbon atoms, 5-6 membered heteroaryl having 1-3 heteroatoms selected from N, NR₄, O, and S;
wherein said alkyl, alkenyl, alkynyl, cycloalkyl, cycloheteroalkyl, aryl and heteroaryl of R₃ may optionally be substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅, perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl; provided that R₃′ does not contain a group that can form an anion under basic conditions;
or a pharmaceutically acceptable salt thereof, which comprises the steps of:
a) reacting a sulfonyl fluoride of formula III
R₃′SO₂F III
wherein R₃′ is as defined in claim 1; with an enol ether of formula VIII: 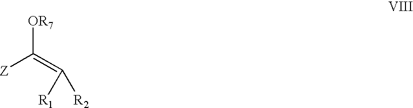
wherein
Z is H, OH, YNOX, OR₅, 13 NR₅R₆ and R₁ and R₂, are as defined in claim 1; and
R₇ is cycloalkyl of 3-6 carbon atoms; 5-10 membered cycloheteroalkyl; alkyl of 1-18 carbon atoms; alkenyl of 2-18 carbon atoms having from 1 to 3 double bonds; alkynyl of 2-18 carbon atoms having from 1 to 3 triple bonds; or —SiR₈R₉R₁₀;
R₈, R₉, and R₁₀ are each, independently, aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms having from 1 to 3 double bonds; alkynyl of 2-18 carbon atoms having from 1 to 3 triple bonds; or two of R₈, R₉, and R₁₀ taken together with the silicon atom to which they are attached form a heterocyclic ring of 5 or 6 members; in the presence of a Lewis acid or fluoride reagent in an ether organic solvent at temperatures ranging from about −78° C. to about 30° C. to produce an alpha-sulfonyl carbonyl compound of formula V; any reactive substituent group(s) being protected during the reaction and removed thereafter; and further if desired isolating any chiral or stereoisomeric product as an individual isomer.

16. The method of claim 15 in which the compound of formula (V) prepared wherein Z is H, OH, —NR₅R₆ or —OR₅ is further reacted to convert it to an alpha-sulfonyl hydroxamic acid derivative of the formula I: 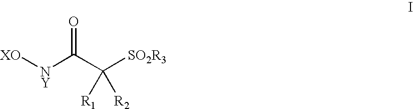
wherein X, Y, R₁, R₂ and R₃ are as defined in claim 1 or a pharmaceutically acceptable salt thereof; any reactive substituent group(s) being protected during the reaction and removed thereafter; and further if desired isolating any chiral or stereoisomeric product as an individual isomer.

17. The method of claim 16 wherein Z in the compound of formula V prepared is:
(i) OR₅ wherein R₅ is other than hydrogen and the conversion to the alpha-sulfonyl hydroxamic acid derivative of the formula I is carried out by:
a) reacting the compound of formula V with an alkali metal hydroxide in the presence of water, and/or ether organic solvent or alcohol at temperatures ranging from about 0° C. to about 100° C. to produce a carboxylic acid of the formula VI: 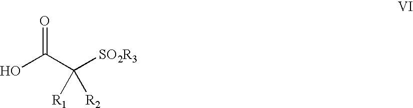
wherein, R₁, R₂, and R₃ are as hereinabove defined; and
(b) reacting the carboxylic acid of formula VI with a hydroxylamine or hydroxylamine derivative of the formula VII:
XONHY VII
wherein X and Y are as hereinabove defined; in the presence of suitable coupling reagent and polar organic solvent to produce a hydroxamate of the formula I or
(ii) OH and the conversion to the alpha-sulfonyl hydroxamic acid derivative of the formula I is carried out according to step b) above.

18. The method of claim 17 wherein the ether organic solvent in step a) is selected from tetrahydrofuran, diethylether and dioxane.

19. The method of claim 17 wherein the alcohol in step a) is selected from methanol and ethanol.

20. The method of claim 17 wherein the alkali metal hydroxide in step a) is selected from lithium hydroxide and sodium hydroxide.

21. The method of claim 17 wherein the polar organic solvent in step b) is dimethylformamide.

22. The method of claim 17 wherein the coupling reagent is selected from the group consisting of 1-(3-dimethylaminopropyl)-3-ethylcarbodimide hydrochloride, N-hydroxybenzotriazole, N-methylmorpholine and oxalylchloride and triethylamine.

23. The method of claim 17 wherein the coupling reaction is carried out at a temperature from about 0° C. to 30° C.

24. The method of claim 15 wherein the Lewis acid or fluoride reagent is selected from boron tribromide, tetrabutyl ammonium fluoride and sodium fluoride.

25. The method of claim 24 wherein the ether organic solvent is selected from tetrahydrofuran, diethylether and dioxane.

26. The method of claim 15 in which the sulfonyl fluoride of formula III is prepared by reacting a sulfonyl chloride of formula II
R₃′SO₂Cl II
wherein R₃′ is as hereinabove defined for R₃ with the proviso that R₃′ does not contain a group that can form an anion under basic conditions, with a fluorinating agent in the presence of a polar organic solvent at from about 15° C. to about 30° C. to produce a sulfonyl fluoride of the formula III.

27. The method of claim 26 wherein the fluorinating agent is selected from the group consisting of potassium fluoride, potassium fluoride-calcium fluoride mixture, and cesium fluoride.

28. The method of claim 26 wherein the polar organic solvent is selected from acetonitrile or tetrahydrofuran.

29. The method of claim 1 wherein X is H or lower alkyl of 1-6 carbon atoms.

30 The method of claim 1 wherein Y is H.

31 The method of claim 1 where Z is OH or OR₅ where R₅ is C₁-C₆ alkyl.

32. The method of claim 1 wherein R₁ and R₂ together form a 5-10 membered cycloheteroalkyl ring containing 1-3 heteroatoms selected from N, NR₄, O and S wherein R₄ is as defined in claim 1.

33. The method of claim 32 wherein the cycloheteroalkyl ring is saturated.

34. The method of claim 32 wherein the cycloheteroalkyl ring is has 6 atoms.

35. The method of claim 32 wherein the heteroatom is NR₄ and R₄ is hydrogen, trifluoromethylsulfonyl, optionally substituted aralkyl of 7-10 carbon atoms, (C₆-C₁₀-aryl)carbonyl-, cycloheteroalkyl-carbonyl or heteroaryl-carbonyl.

36. The method of claim 1 wherein R₃ is an optionally substituted C₆-C₁₀ aryl group.

37. The method of claim 1 wherein R₃ is a phenyl group substituted by one or more OR₅ groups.

38. The method of claim 1 wherein R₅ is C₁-C₆ alkyl, C₂-C₆ alkenyl, C₂-C₆ alkynyl or halophenyl.

39 The method claim 1 in which the compound prepared is an alpha-sulfonyl hydroxamic acid derivatives of the general formula IA: 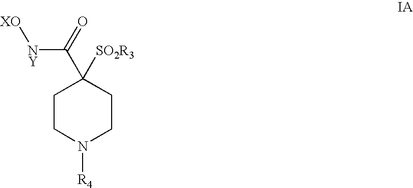
wherein
X is hydrogen, or alkyl of 1-6 carbon atoms; and Y, R₃ and R₄ are as defined in claim 1 or a pharmaceutically acceptable salt thereof;

40. A method of preparing alpha-sulfonyl hydroxamic acid derivatives of the general formula IA: 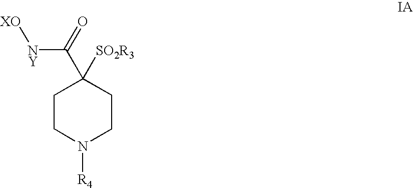
wherein
X is hydrogen, or alkyl of 1-6 carbon atoms; and Y, R₃ and R₄ are as defined in claim 1 or a pharmaceutically acceptable salt thereof;
which comprises:
a) treating a compound of formula 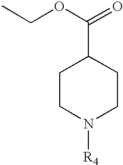
with diisopropylamide or lithium hexamethyldisilazide to form an enolate;
b) reacting the enolate with a sulfonyl fluoride:
R₃SO₂F
to form a compound 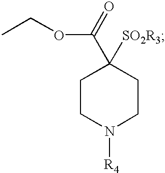
c) hydrolyzing the compound of step b) to produce 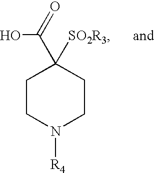
d) reacting compound of step c) with hydroxylamine or hydroxylamine derivative of the formula:
XONHY
in the presence of coupling reagent and polar organic solvent at temperatures ranging from about 0° C. to about 30° C.; and if desired isolating as a pharmaceutically acceptable salt.

41. The method of claim 40 wherein the coupling reagent is selected from 1-(3-dimethylaminopropyl)-3-ethylcarbodimide hydrochloride, N-hydroxybenzotriazole, N-methylmorpholine and oxalylchloride and triethylamine.

42. The method of claim 41 wherein the polar organic solvent is dimethylformamide.

43. A method of preparing a compound of the formula 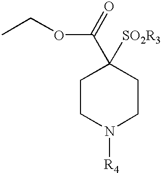
wherein
R₃ and R₄ are as defined in claim 1 or a pharmaceutically acceptable salt thereof, which comprises the steps of:
a) treating a compound of formula 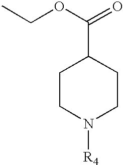
with diisopropylamide or lithium hexamethyldisilazide to form an enolate; and
b) reacting the enolate with a sulfonyl fluoride of formula:
R₃SO₂F

44. A method of preparing a compound of Formula 8 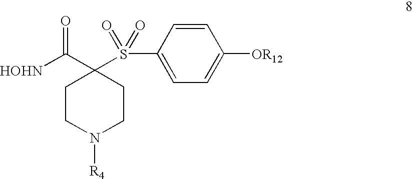
wherein R₄ is as defined in claim 1 and R₁₂ is methyl, n-butyl, 2-butynyl, or p-chlorophenyl; and n is 1 or 2; or a pharmaceutically acceptable salt thereof, which comprises the steps of:
a) treating a compound of formula 12 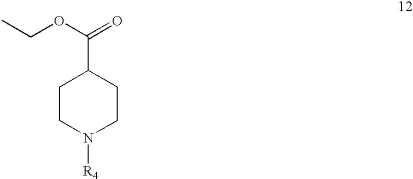
with diisopropylamide or lithium hexamethyldisilazide to form an enolate;
b) reacting the enolate with a sulfonyl fluoride of Formula 2: 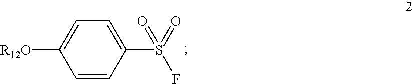
to form a compound of Formula 13 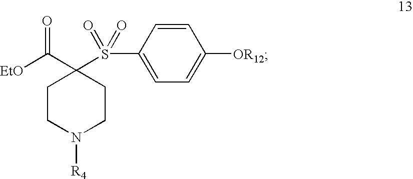
c) hydrolyzing compound of Formula 13 with lithium hydroxide to produce compound of Formula 14 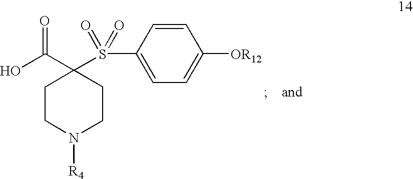
d) treating the compound of Formula 14 with oxalyl chloride, triethylamine, and hydroxylamine hydrochloride at temperatures ranging from about 0° C. to about 30° C.

45. A compound of Formula IX 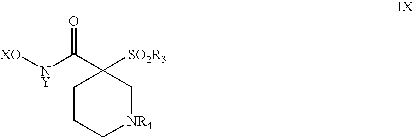
wherein
X is hydrogen, or alkyl of 1-6 carbon atoms;
Y is hydrogen, alkyl of 1-6 carbon atoms, aryl of 6 to 10 carbon atoms, 5-10 membered heteroaryl having 1-3 heteroatoms selected from N, NR₄, O and S, cycloalkyl of 3-6 carbon atoms, 5-10 membered cycloheteroalkyl; wherein said alkyl, aryl, heteroaryl, cycloalkyl and cycloheteroalkyl group of Y is optionally substituted on any atom capable of substitution, with 1 to 3 substituents selected from the group consisting of halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅ , perfluoroalkyl of 1-4 carbon atoms, —O-perfluoroalkyl of 1-4 carbon atoms, —CONR₅R₆, —S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, 13 COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆, —NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl; R₃ is alkyl of 1-18 carbon atoms, alkenyl of 2-18 carbon atoms having 1 to 3 double bonds, alkynyl of 2-18 carbon atoms having from 1 to 3 triple bonds, cycloalkyl of 3-6 carbon atoms, 5-10 membered cycloheteroalkyl, aryl of 6 to 10 carbon atoms, 5-6 membered heteroaryl having 1-3 heteroatoms selected from N, NR₄, O and S; wherein said alkyl, alkenyl, alkynyl, cycloalkyl, cycloheteroalkyl, aryl and heteroaryl of R₃ may optionally be substituted on any atom capable of substitution with from 1 to 3 substituents selected from halogen, alkyl of 1-6 carbon atoms; alkenyl of 2-6 carbon atoms having from 1 to 3 double bonds; alkynyl of 2-6 carbon atoms having from 1 to 3 triple bonds, cycloalkyl of 3-6 carbon atoms, —OR₅, ═O, —CN, —COR₅, perfluoroalkyl of 1-4 carbon atoms, —O-perfluoroalkyl of 1-4 carbon atoms, —CONR₅R₆,—S(O)ₙR₅, —OPO(OR₅)OR₆, —PO(OR₅)R₆, —OC(O)OR₅, —OR₅NR₅R₆, —OC(O)NR₅R₆, —C(O)NR₅OR₆, —COOR₅, —SO₃H, —NR₅R₆, —N[(CH₂)₂]₂NR₅, —NR₅COR₆, —NR₅COOR₆, SO₂NR₅R₆, —NO₂, —N(R₅)SO₂R₆—NR₅CONR₅R₆, —NR₅C(═NR₆)NR₅R₆, —NR₅C(═NR₆)N(SO₂R₅)R₆, —NR₅C(═NR₆)N(C═OR₅)R₆, -tetrazol-5-yl, —SO₂NHCN, —SO₂NHCONR₅R₆, phenyl, heteroaryl and 5-10 membered cycloheteroalkyl;
R₄ is hydrogen; aryl; aralkyl, heteroaryl; heteroaralkyl, alkyl of 1-6 carbon atoms; cycloalkyl of 3-6 carbon atoms; —C(O)ₙR₅, —CONR₅R₆ or SO₂R₅;
R₅ and R₆ are each independently hydrogen, optionally substituted aryl; 4-8 membered heteroaryl having 1-3 heteroatoms selected from N, NR₄, O and S; cycloalkyl of 3-6 carbon atoms; 5-10 membered cycloheteroalkyl; alkyl of 1-18 carbon atoms; alkenyl of 2-18 carbon atoms or alkynyl of 2-18 carbon atoms; or R₅ and R₆ taken together with the nitrogen atom to which they are attached may form a 5-10 membered cycloheteroalkyl ring; and
n is 1 or 2; or an optical isomer thereof or a pharmaceutically acceptable salt thereof.

46. A compound according to claim 45 which is 1-benzyl-3-(4-methoxy-benzenesulfonyl)piperidine-3-carboxylic acid hydroxamide.

47. A pharmaceutical composition comprising a compound of Formula IX 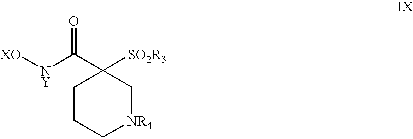
as defined in claim 45 or claim 46 or a pharmaceutically acceptable salt thereof; and a pharmaceutically acceptable carrier.

48. A method of inhibiting pathological changes mediated by TNF-alpha converting enzymes (TACE) in a mammal in need thereof which comprises administering to said mammal a therapeutically effective amount of a compound of claim 45, or a pharmaceutically acceptable salt thereof.

49. The method of claim 48 wherein the condition treated is rheumatoid arthritis, graft rejection, cachexia, inflammation, fever, insulin resistance, septic shock, congestive heart failure, inflammatory disease of the central nervous sytem, inflammatory bowel disease or HIV infection.

50. A method of inhibiting pathological changes mediated by matrix metalloproteinases in a mammal in need thereof which comprises administering to said mammal a therapeutically effective amount of a compound of claim 45, or a pharmaceutically acceptable salt thereof.

51. The method of claim 50 wherein the condition treated is age related macular degeneration, diabetic retinopathy, proliferative vitreoretinopathy, retinopathy of prematurity, ocular inflammation, keratoconus, Sjogren's syndrome, myopia, ocular tumors, ocular angiogenesis/neovascularization and corneal graft rejection.

52. The method of claim 50 wherein the condition treated is atherosclerosis, atherosclerotic plaque formation, reduction of coronary thrombosis from atherosclerotic plaque rupture, restenosis, MMP-mediated osteopenias, inflammatory diseases of the central nervous system, skin aging, angiogenesis, tumor metastasis, tumor growth, osteoarthritis, rheumatoid arthritis, septic arthritis, corneal ulceration, abnormal wound healing, bone disease, proteinuria, aneurysmal aortic disease, degenerative cartilage loss following traumatic joint injury, demyelinating diseases of the nervous system, cirrhosis of the liver, glomerular disease of the kidney, premature rupture of fetal membranes, inflammatory bowel disease, or periodontal disease.
